SEQUENCE LISTING (43 sequences)

SEQ ID NO: 1 - protein, 250 aa, synthetic construct
MDPRLREEVVRLIIALTSDNGVSLSKGLESRVSALEKTSQIHSDTILRITQGLDDANKRIIALEQSRDDLVASVSDAQLAISRLESSIGALQTVVNGLDSSVTQLGARVGQLETGLAELRVDHDNLVARVDTAERNIGSLTTELSTLTLRVTSIQADFESRISTLERTAVTSAGAPLSIRNNRMTMGLNDGLXLSGNNLAIRLPGNTGLNIQNGGLQFRFNTDQFQIVNNNLTLKTTVFDSINSRXGAXE

SEQ ID NO: 2 - protein, 250 aa, synthetic construct
MDPRLREEVVRLIIALTSDNGASLSKGLESRVSALEKTSQIHSDTILRITQGLDDANKRIIALEQSRDDLVASVSDAQLAISRLESSIGALQTVVNGLDSSVTQLGARVGQLETGLAELRVDHDNLVARVDTAERNIGSLTTELSTLTLRVTSIQADFESRISTLERTAVTSAGAPLSIRNNRMTMGLNDGLTLSGNNLAIRLPGNTGLNIQNGGLQFRFNTDQFQVVNNNLTLKTTVFDSINSRIGATE

SEQ ID NO: 3 - protein, 455 aa, unidentified
  source 1..455: note = Reoviridae
MDPRLREEVVRLIIALTSDNGVSLSKGLESRVSALEKTSQIHSDTILRITQGLDDANKRIIALEQSRDDLVASVSDAQLAISRLESSIGALQTVVNGLDSSVTQLGARVGQLETGLAELRVDHDNLVARVDTAERNIGSLTTELSTLTLRVTSIQADFESRISTLERTAVTSAGAPLSIRNNRMTMGLNDGLXLSGNNLAIRLPGNTGLNIQNGGLQFRFNTDQFQIVNNNLTLKTTVFDSINSRXGAXEQSXVASAVTPLRLNSSTKVLDMLIDSSTLEINSSGQLTVRSTSPNLRYPIADVSGGIGMSPNYRFRQSMWIGIVSYSGSGLNWRVQVNSDIFIVDDYIHICLPAFDGFSIADGGDLSLNFVTGLLPPLLTGDTEPAFHNDVVTYGAQTVAIGLSSGGAPQYMSKNLWVEQWQDGVLRLRVEGGGSITHSNSKWPAMTVSYPRSFT

SEQ ID NO: 4 - DNA, 765 bp, synthetic construct
  source 1..765: mol_type = other DNA
gctattggtcggatggatcctcgcctacgtgaagaagtagtacggctgataatcgcattaacgagtgataatggagtatcactgtcaaaagggcttgaatcaagggtctcggcgctcgagaagacgtctcaaatacactctgatactatcctccggatcacccagggactcgatgatgcaaacaaacgaatcatcgctcttgagcaaagtcgggatgacttggttgcatcagtcagtgatgctcaacttgcaatctccagattggaaagctctatcggagccctccaaacagttgtcaatggacttgattcgagtgttacccagttgggtgctcgagtgggacaacttgagacaggacttgcagagctacgcgttgatcacgacaatctcgttgcgagagtggatactgcagaacgtaacattggatcattgaccactgagctatcaactctgacgttacgagtaacatccatacaagcggatttcgaatctaggatatccacattagagcgcacggcggtcactagcgcgggagctcccctctcaatccgtaataaccgtatgaccatgggattaaatgatggactcaygttgtcagggaataatctcgccatccgattgccaggaaatacgggtctgaatattcaaaatggtggacttcagtttcgatttaatactgatcaattccagatagttaataataacttgactctcaagacgactgtgtttgattctatcaactcaaggabaggcgcaaytgagtaa

SEQ ID NO: 5 - DNA, 1416 bp, synthetic construct
  source 1..1416: mol_type = other DNA
gctattggtcggatggatcctcgcctacgtgaagaagtagtacggctgataatcgcattaacgagtgataatggagtatcactgtcaaaagggcttgaatcaagggtctcggcgctcgagaagacgtctcaaatacactctgatactatcctccggatcacccagggactcgatgatgcaaacaaacgaatcatcgctcttgagcaaagtcgggatgacttggttgcatcagtcagtgatgctcaacttgcaatctccagattggaaagctctatcggagccctccaaacagttgtcaatggacttgattcgagtgttacccagttgggtgctcgagtgggacaacttgagacaggacttgcagagctacgcgttgatcacgacaatctcgttgcgagagtggatactgcagaacgtaacattggatcattgaccactgagctatcaactctgacgttacgagtaacatccatacaagcggatttcgaatctaggatatccacattagagcgcacggcggtcactagcgcgggagctcccctctcaatccgtaataaccgtatgaccatgggattaaatgatggactcaygttgtcagggaataatctcgccatccgattgccaggaaatacgggtctgaatattcaaaatggtggacttcagtttcgatttaatactgatcaattccagatagttaataataacttgactctcaagacgactgtgtttgattctatcaactcaaggabaggcgcaaytgagtaaagtkmcgtggcgtcggcagtgactcccttgagattaaacagtagcacgaaggtgctggatatgctaatagacagttcaacacttgaaattaattctagtggacagctaactgttagatcgacatccccgaatttgaggtatccgatagctgatgttagcggcggtatcggaatgagtccaaattataggtttaggcagagcatgtggataggaattgtctcctattctggtagtgggctgaattggagggtacaggtgaactccgacatttttattgtagatgattacatacatatatgtcttccagcttttgacggtttctctatagctgacggtggagatctatcgttgaactttgttaccggattgttaccaccgttacttacaggagacactgagcccgcttttcataatgacgtggtcacatatggagcacagactgtagctatagggttgtcgtcgggtggtgcgcctcagtatatgagtaagaatctgtgggtggagcagtggcaggatggagtacttcggttacgtgttgaggggggtggctcaattacgcactcaaacagtaagtggcctgccatgaccgtttcgtacccgcgtagtttcacgtgaggatcagaccaccccgcggcactggggcatttcatc

SEQ ID NO: 6 - DNA, 1416 bp, unidentified
  source 1..1416: mol_type = other DNA; note = Reoviridae
gctattggtcggatggatcctcgcctacgtgaagaagtagtacggctgataatcgcattaacgagtgataatggagtatcactgtcaaaagggcttgaatcaagggtctcggcgctcgagaagacgtctcaaatacactctgatactatcctccggatcacccagggactcgatgatgcaaacaaacgaatcatcgctcttgagcaaagtcgggatgacttggttgcatcagtcagtgatgctcaacttgcaatctccagattggaaagctctatcggagccctccaaacagttgtcaatggacttgattcgagtgttacccagttgggtgctcgagtgggacaacttgagacaggacttgcagagctacgcgttgatcacgacaatctcgttgcgagagtggatactgcagaacgtaacattggatcattgaccactgagctatcaactctgacgttacgagtaacatccatacaagcggatttcgaatctaggatatccacattagagcgcacggcggtcactagcgcgggagctcccctctcaatccgtaataaccgtatgaccatgggattaaatgatggactcaygttgtcagggaataatctcgccatccgattgccaggaaatacgggtctgaatattcaaaatggtggacttcagtttcgatttaatactgatcaattccagatagttaataataacttgactctcaagacgactgtgtttgattctatcaactcaaggabaggcgcaaytgagcaaagtkmcgtggcgtcggcagtgactcccttgagattaaacagtagcacgaaggtgctggatatgctaatagacagttcaacacttgaaattaattctagtggacagctaactgttagatcgacatccccgaatttgaggtatccgatagctgatgttagcggcggtatcggaatgagtccaaattataggtttaggcagagcatgtggataggaattgtctcctattctggtagtgggctgaattggagggtacaggtgaactccgacatttttattgtagatgattacatacatatatgtcttccagcttttgacggtttctctatagctgacggtggagatctatcgttgaactttgttaccggattgttaccaccgttacttacaggagacactgagcccgcttttcataatgacgtggtcacatatggagcacagactgtagctatagggttgtcgtcgggtggtgcgcctcagtatatgagtaagaatctgtgggtggagcagtggcaggatggagtacttcggttacgtgttgaggggggtggctcaattacgcactcaaacagtaagtggcctgccatgaccgtttcgtacccgcgtagtttcacgtgaggatcagaccaccccgcggcactggggcatttcatc

SEQ ID NO: 7 - DNA, 3860 bp, unidentified
  source 1..3860: mol_type = other DNA; note = Reoviridae
gctacacgttccacttccacgacaatgtcatccatgatactgactcagtttggaccgttcattgagagcatttcaggtatcactgatcaatcgaatgacgtgtttgaagatgcagcaaaagcattctctatgtttactcgcagcgatgtctacaaggcgctggatgaaatacctttctctgatgatgcgatgcttccaatccctccaactatatatacgaaaccatctcacgattcatattattacattgatgctctaaaccgtgtgcgtcgcaaaacatatcagggccctgatgacgtgtacgtacctaattgttctattgttgaattgctggagccacatgagactctgacatcttatgggcggttgtccgaggccatcgagaatcgtgccaaggatggggacagccaagccagaatcgccacaacgtatggtagaatcgctgaatctcaagctcgacagattaaggctccattggagaagtttgtgttggcactattagtggccgaagcaggggggtctttatatgatccagttttgcagaagtatgatgagattccagatctatcgcataattgccctttatggtgttttagagagatctgtcgtcacatatctggtccattaccagatcgggcaccttatctttacttatctgcaggggttttctggttaatgtcaccacgaatgacgtctgcaatccctccgctactatccgatcttgttaatttagctattttgcaacaaactgcgggtttagatccatcattagtgaaattgggagtacagatatgccttcatgcagcagctagctcaagttatgcatggtttatcttaaagactaagtctatttttcctcaaaacacgttgcacagtatgtatgaatctctagaagggggatactgtcctaatcttgaatggttagagcctagatcagactataagttcatgtacatgggagtcatgccattgtccgctaagtatgctaggtcggcgccgtccaatgataagaaagcgcgggaacttggcgagaaatatggactgagctcagtcgtcggtgagcttcgtaaacggacaaagacgtatgttaaacatgactttgcttcagtgaggtacattcgtgacgctatggcatgtactagcggtattttcttggtaagaacacccaccgaaacggtattgcaagaatatacgcagagtccggagattaaggttcccattccccagaaagactggacaggcccaataggtgaaatcagaattctaaaagatacaacaagttccatcgcgcgttacttatatagaacatggtacttggcagcggcgagaatggcggctcaaccacgtacgtgggatccattgtttcaagcgattatgagatctcaatacgtgacagctaggggtggatctggcgcagcactccgcgaatctttgtatgcaatcaatgtgtcgttacctgatttcaagggcttaccagtgaaggcagcaactaagatattccaggcggcacaattagcgaacttgccgttctcccacacatcagtggctatactagctgacacttcaatgggattgcgaaatcaggtgcagaggcggccacgatccattatgccattaaatgtgccccagcagcaggtttcggcgccccatacattgacagcggattacattaactaccacatgaatctatcaaccacgtctggtagtgcggtcattgagaaggtgattcctttaggtgtatacgcttcgagccctcctaaccagtcgatcaacattgacatatctgcgtgtgacgctagtattacttgggatttctttctgtcagtgattatggcggctatacacgaaggtgtcgctagtagctccattggaaaaccatttatgggggttcctgcatccattgtaaatgatgagtctgtcgttggagtgagagctgctaggccgatatcgggaatgcagaacatgattcagcatctatcgaaactatataaacgtggattttcatatagagtaaacgattctttttctccaggtaacgattttactcatatgactaccactttcccgtcaggttcaacagccacctctactgagcatactgctaataatagtacgatgatggaaactttcctgacagtatggggacccgaacatactgacgaccctgacgtcttacgtttaatgaagtctttaactattcaaaggaattacgtatgtcaaggtgatgatggattaatgattatcgatgggactactgctggtaaggtgaacagtgaaactattcagaagatgctagaattaatctcaaaatatggtgaggaattcggatggaaatatgacatagcgtacgatgggactgccgaatacttaaagctatacttcatatttggctgtcgaattccaaatcttagtcgccatccaatcgtggggaaagaacgggcgaattcttcagcagaggagccatggccagcaattctagatcagattatgggtgtcttctttaatggtgttcatgatgggttacagtggcagcggtggatacgttattcatgggctctatgctgtgctttctcacgtcaaagaacaatgattggtgagagcgtgggttaccttcaatatcctatgtggtcttttgtctactggggattaccactggttaaagcgtttgggtcagacccatggatattttcttggtacatgcctactggagatctgggaatgtatagttggattagcttgatacgccctctgatgacaagatggatggtggctaatggttacgtaactgacagatgctcacccgtattcgggaacgcagattatcgcaggtgtttcaatgaacttaaactatatcaaggttattatatggcacaattgcccaggaatcctaagaagtctggacgagcggcccctcgggaggtaagagaacaattcactcaggcattatccgactatctaatgcaaaatccagagctgaagtcacgtgtgctacgtggtcgtagtgagtgggagaaatatggagcggggataattcacaatcctccgtcattattcgatgtgccccataaatggtatcagggtgcgcaagaggcagcaatcgctacgagagaagagctggcagaaatggatgagacattaatgcgcgctcgaaggcacagctattcgagcttttcaaagttattagaggcgtatctgctcgtgaaatggcgaatgtgcgaggcccgcgaaccgtcggttgatttgcgattaccattatgtgcgggtattgacccattaaactcagatccttttctcaagatggtaagcgttggaccaatgctccagagtacgagaaagtactttgctcagacactattcatggcaaagacggtgtcgggtcttgacgttaacgcgattgatagcgcgttattacgactgcgaacattaggtgctgataagaaagcattaacggcgcagttattaatggtggggcttcaggagtcagaagcggacgcattggccgggaagataatgctacaggatgtgaatactgtgcaattagccagagtggttaacttagctgtgccagatacttggatgtcgttagactttgaytctatgttcaaacaccacgtcaagctgcttcccaaagatggacgtcatctaaatactgatattcctcctcgaatgggatggttacgggccattttacgattcttaggtgccggaatggtaatgactgcgactggagttgctgtcgacatctatctggaggatatacatggcggtggtcggtcacttggacagagattcatgacttggatgcgacaggaaggacggtcagcgtgagtctaccatgggtcgtggtgcgtcaactcatc

SEQ ID NO: 8 - DNA, 3916 bp, unidentified
  source 1..3916: mol_type = other DNA; note = Reoviridae
gctaaaaggcgcgatggcgaacgtttggggggtgagacttgcagactcgttatcttcacccactattgagacacgaacgcgtcagtataccttacacgatctttgctcagacctagatgctaatccggggagggaaccgtggaaacctctgcgtaatcagcgtactaataatattgtggctgtgcaattattcagaccattgcagggtttagttttagatacccagctttatggatttccaggagcatttgatgactgggagcgattcatgagagagaagctgcgtgtgctaaagtatgaagtattgcgcatctatccaatcagcaactatagcaatgaacatgtcaacgtcttcgtggccaatgctttggtgggcgctttcctgtcgaatcaagctttctatgacctgctaccgttgttgataattaatgacactatgattggtgatctacttggcacgggggcatcgctatcacagttctttcaatctcatggagatgtgctggaagtcgcagctggtcgtaagtatctgcagatggaaaactactccaacgatgacgatgatcctccattatttgcgaaagacctgtcagattatgctaaagcattctacagtgacacatatgaagtgttggacaggttcttttggacgcatgactcttcagcgggggtcttagtgcattatgataagccaacgaatggtcatcactatctgctgggtactttgactcagatggtcagtgcacctccttatattattaacgctactgacgcaatgttgcttgaatcctgtctagaacagttctcagctaatgtgcgtgcgagacctgcgcaacccgttacacgcttagaccaatgctatcatttaagatggggagcacaatatgtaggagaagattcactgacatatcggttgggggtgttatccttgctggctaccaatggatatcaattagctagaccgattccaagacagttgacgaatcgatggttgtcgagctttgtgagtcaaattatgtctgacggcgtcaacgagactccactgtggccccaagaaaggtatgtgcagatcgcttatgattcaccatccgttgttgatggggctacgcaatatggctatgtcaggaagaatcaactcagactcggcatgagaatatcggcgctgcaatcgctgagtgatacgccctcgccggtacagtggcttccacaatacaccatcgaccaggcagcgatggacgaaggcgatctgatggttagtcggcttacgcaactcccgttacgtcctgattatggtaatatctgggtcggcgatgcgctatcctattatgtggactacaatcggagtcatcgagtcgtgctttcatcggaacttcctcagcttccggacacatattttgatggcgatgaacagtatgggcgcagcctgttctcactagctcgtaagattggtgaccgctcgttagtgaaagatacggctgtcttgaagcacgcttaccaagccatcgatccaaatactggtaaggagtatctgagatctgggcaatctgtcgcatattttggtgcatcagcgggtcattctggtgccgaccagccgttagtcatagagccctggattcaagggaaaatcagtggtgtgccgccaccctcctcagtgcgacagttcggctatgatgttgcccgtggcgcgatcgtcgatctggcgagaccatttccttctggagattatcaatttgtctattcggatgttgaccaggtggtcgatggccatgacgatctgagtatatcatctggactggtggagagccttttgtcttcatgcatgcacgccacagcacccgggggctcatttgttgttaagataaattttccgactagacccgtatggcactacatcgaacagaagatcttgcccaatattacgtcatacatgttgatcaagcctttcgtcaccaacaacgtcgaattgttcttcgtcgctttcggtgtgcatcaacactcatcacttacttggacatctggagtgtacttcttcttggtggaccatttttatcgttatgagactttatctacgatctcacgacaattgccgtcttttgggtatgttgatgatgggtcttccgtgactggtatcgagacaattagtattgagaaccctggcttctcgaatatgacccaggccgctcgcattggtatctcaggattgtgtgctaatgtaggtaacgcgcgtaagtccattgccatttacgaatcccatggggccagagtattaactatcacatcaaggagatctccggcatcagctagaagaaagtctaggttgcgatatttgccattaatagaccctaggtcgttagaggtacaggcgcgcactattctgccagctgatccagtgttatttgaaaacgtgagcggagcgtcaccccatgtttgtctgacaatgatgtacaacttcgaagtgtcgtcagcggtatatgatggagacgttgtgctagatcttgggacgggaccagaggctaaaatccttgaactgatacccgcaacctctccagtcacatgcgtggacatacggcctacagcgcagcctagtggatgttggaacgttcgtaccacgttccttgagttagattatttgagcgatggatggatcactggggtgcgtggggacatagttacttgtatgttatctttgggggccgctgccgctggaaaatcaatgacttttgacgctgcgtttcagcaattaatcaaagtattatccaagagtacggctaatgttgtgctggtgcaggttaactgccctacagacgtggtgaggagcattaagggctacctagagatagattcgactaacaagaggtataggttccccaaatttggtcgagacgagccgtactctgacatggatgcgctggagaaaatatgtcgtaccgcctggccaaactgctcaattacctgggttccattgtcatacgacttgcggtggactagactggcattattagagtccacgacattgagtagcgcgtcgattagaattgctgagctgatgtataaatacatgcctattatgaggattgatattcatggactacccatggaaaagcgaggtaacttcatagtggggcagaactgctcattagtaatccctggttttaatgcgcaggatgtctttaactgttatttcaattccgccctcgctttctcgactgaagatgtcaatgctgcgatgattccccaagtgtctgcgcagtttgatgcgactaagggtgagtggacgttggatatggtcttctccgacgcaggaatctataccatgcaggctctagtgggatctaatgctaatccagtctctttgggttcctttgtagttgattctccagatgtagatataactgacgcttggccagctcagttagactttacgatcgcgggaactgatgtcgatataacagttaatccttattaccgtctgatgacctttgtaaggatcgatggacagtggcagattgccaatccagacaaatttcaattcttttcgtcggcgtctgggacgttagtgatgaacgtcaaattagatatcgcagataaatatctactatactatatacgagatgtccagtctcgagatgttggcttttacattcagcatccacttcaacttttgaatacgatcacattgccaaccaacgaggacctttttctgagcgcacctgacatgcgagagtgggcagttaaggaaagcggtaacacgatatgtatactcaatagtcaagggtttgtgctacctcaagattgggatgtgttaacagataccataagttggtccccatcgatacccacatacattgtgccaccgggtgattataccttgactcctctgtaactcactgtccctcgtgagcgcgcctaattcatc

SEQ ID NO: 9 - DNA, 3901 bp, unidentified
  source 1..3901: mol_type = other DNA; note = Reoviridae
gctaatcgtcaggatgaagcggattccaaggaagacaaagggcaaatccagcggaaagggcaatgactcaacagagagagcggacgatggctcgagccaattaagagacaagcaaaacaataaggctggccccgccactacggagcctggcacatccaaccgagagcaatacaaagctcgaccaggtattgcatctgtgcagagggccactgaaagtgcagaaatgcccatgaagaataatgacgaagggacgccagataagaaaggaaatactaagggcgacctagttaatgagcatagtgaggctaaagacgaggcggatgaagcgacgaagaagcaggcaaaggatacagacaaaagtaaagcgcaagtcacatattcagacactggtatcaataatgctaatgaactgtcaagatctgggaatgtggataatgagggtggaagtaatcagaagccgatgtctaccagaatagctgaggcaacgtctgctatagtgtcgaaacatcctgcgcgtgttgggctgccacctaccgctagcagtggtcatgggtatcagtgccatgtctgttctgcagtcctgtttagtcctttagacctagatgcccacgtcgcctcacatggtttgcatggtaacatgacattaacatcgagtgatatccagcgacatataactgagttcatcagctcatggcaaaatcatcctattgttcaagtttcggctgatgtcgaaaataagaaaactgctcaattgcttcacgctgacactcctcgactcgtcacttgggatgctggtttgtgtacttcattcaaaatcgtcccgattgtgccagctcaggtgccgcaggatgtactggcctatacgtttttcacctcttcatacgctatccaatcaccgtttccagaggcggcagtgtctaggattgtggtgcatacgagatgggcatctaatgttgactttgaccgagactcgtctgtcatcatggcgccacctacagaaaacaatatccatttgtttaaacagttactaaatactgaaaccctgtctgtaaggggggctaatccgctaatgttcagggcgaatgtgttgcatatgttgctagagttcgtattagataacttgtatctgaacagacatacgggattctctcaagaccacacgccatttactgagggtgctaatttgcgttcacttcctggccccgatgctgagaaatggtactcgattatgtatccaacgcgcatgggaacgccgaatgtatccaaaatatgtaatttcgtcgcctcttgtgtgcgaaatcgggttggacggtttgatcgagcacagatgatgaacggagctatgtcagagtgggtggatgtcttcgagacttcagacgcgctaaccgtctccattcgaggtcgatggatggctagactagctcgcatgaacataaatccaacagagatcgaatgggcattgactgaatgtgcacaaggatatgtgactgtcacaagtccttacgctcctagcgtaaatagattgatgccctatcgtatctccaacgctgagcggcaaatatcacagataatcaggatcatgaacattggcaataacgcgacggtgatacaacctgttctgcaagatatttcggtactccttcaacgcatatcaccactccaaatagatccaactattatttccaacactatgtcaacagtctcggagtctactactcagaccctcagccccgcgtcctcaattttgggtaaactacgaccaagcaactcagatttttctagttttagagtcgcgttggctggatggctttataatggggttgtgacgacggtgattgatgatagttcatatccaaaagacggcggcagcgtgacctcacttgaaaatctgtgggatttcttcatccttgcgcttgctctaccactgacaactgacccctgtgcacctgtgaaagcattcatgaccctagccaacatgatggttggtttcgagacaatccctatggataatcagatctatactcaatcgagacgcgcgagtgctttctcaacgcctcacacgtggccacgatgctttatgaacatccagttaatttctccaatcgacgctcccatcttgcgacagtgggctgaaattattcatagatactggcctaacccttcacagattcgttatggtgcaccgaacgttttcggctcggcaaatttgttcactccacctgaggtgctgttattgccaatcgatcatcaaccagctaatgtaacaacgccaacgctggacttcaccaatgagttaactaattggcgcgctcgtgtctgtgagcttatgaagaatctcgttgataaccaaagatatcaacctggatggacacaaagtctagtctcgtcaatgcgcggaacgctagacaaattgaagttgattaaatcgatgacaccaatgtatctgcaacagctggctccggtagagttagcagtgatagctcccatgttgccttttccacctttccaggtgccatacgtccgtctcgatcgtgacagagttccaacaatggttggagtaacacgacagtcacgagatactattactcagccggcgctatcgctgtcgacaaccaatactactgttggcgtgccactagctctagacgcgagggctatcaccgttgcgctgttgtcagggaaatatccgccggatttggtgacaaatgtatggtacgctgatgccatttacccaatgtatgcagacacggaggtgttctctaatcttcagagagacatgattacctgcgaggccgtgcagacattagtgactctggtggcgcaaatatcagagacccagtatcctgtagataggtatcttgattggatcccatcactgagagcatcggcggcgacggcggcgacatttgctgagtgggttaatacttcaatgaagacggcgtttgatttgtctgatatgctgttagagcctctcctaagcggtgatccgaggatgactcaactagcgattcagtatcagcagtacaatggcagaacgtttaatatcatacctgaaatgccaggttcagtaattgctgactgcgttcaattaacagcagaagtctttaatcacgaatataacctgtttgggattgcgcggggtgatatcatcattggccgtgttcagtcgacacatttgtggtcaccgctggctcctccacctgacctggtgtttgatcgtgatacccctggtgttcacatcttcggacgagattgccgtatatcgtttggaatgaatggcgccgcgccaatgattagagatgagactggactgatggtgccttttgaaggaaattggattttcccactggcgctttggcaaatgaatacacgatattttaatcaacagttcgacgcgtggattaagacaggagagttgcgaatccgcattgagatgggcgcgtatccatatatgttgcattactatgatccacgtcagtacgctaatgcatggaatttaacatccgcctggcttgaagaaattacgccgacgagcatcccatccgtgcctttcatggtgcccatttcaagtgatcatgacatttcctctgccccagctgtccaatatatcatttcaactgaatataatgatcggtctctgttctgcactaactcatcatctccccaaaccatcgctggaccagacaaacacattccagttgagagatataacattctgaccaaccccgacgctccacccacgcagatacaactgcctgaagtcgttgacttgtacaacgtcgtcacacgctatgcgtatgagactccgcctattaccgctgttgttatgggtgttccttgatcctcatcctcccaacaggtgctagagcattgcgctcaatgctagttgggccgattcatc

SEQ ID NO: 10 - DNA, 2224 bp, unidentified
  source 1..2224: mol_type = other DNA; note = Reoviridae
cgtttggagtagacgctggggctgacgcgaatgacgtttcatatcaagatcatgactatgtgttggatcagttacagtacatgttagatggatatgaggctggtgacgttatcgatgcactcgtccacaagaattggttacatcactctgtctattgcttgttgccacccaaaagtcaactattagagtattggaaaagtaatccttcagcgataccggacaacgttgatcgtcggcttcgtaaacgactaatgctaaagaaagatctcaggaaagatgatgaatacaatcagctagcgcgtgctttcaagatatcggatgtctacgcacctctcatctcatccacgacgtcaccgatgacaatgatacagaacttgaatcaaggcgagatcgtgtacaccacgacggacagggtaataggggctagaatcttgttatatgctcctagaaagtactatgcgtcaactctgtcatttactatgactaagtgcatcattccgtttggtaaagaggtgggtcgtgttcctcactctcgatttaatgttggcacatttccgtcaattgctaccccgaaatgttttgtcatgagtggggttgatattgagtccatcccaaatgaatttatcaagttgttttaccagcgcgtcaagagtgttcacgctaacatactaaatgacatatctcctcagatcgtctctgacatgataaacagaaagcgtctgcgcgttcatactccatcagatcgtcgagccgcgcagttgatgcatttgccttaccatgttaaacgaggagcgtctcacgtcgacgtttacaaggtggatgttgtagacatgttgttcgaggtagtggatgtggccgatgggttgcgcaacgtatctaggaaactaactatgcataccgttccggtatgtattcttgaaatgttgggtattgagattgcggactattgcattcgtcaagaggatggaatgctcacagattggttcctacttttaaccatgctatctgatggcttgactgatagaaggacgcattgtcaatacttgattaatccgtcaagtgtgcctcctgatgtgatacttaacatctcaattactggatttataaatagacatacaatcgatgtcatgcctgacatatatgacttcgttaaacccattggcgctgtgctgcctaagggatcatttaaatcaacaattatgagagttcttgattcaatatcaatattaggaatccaaatcatgccgcgcgcgcatgtagttgactcagatgaggtgggcgagcaaatggagcctacgtttgagcaggcggttatggagatatacaaagggattgctggcgttgactcgctggatgatctcatcaagtgggtgctgaactcggatctcattccgcatgatgacaggcttggtcaattatttcaagcgtttttgcctctcgcaaaggacttattagctccaatggccagaaagttttatgataactcaatgagtgagggtagattgctaacattcgctcatgccgacagtgagttgctgaacgcaaattattttggtcatttattgcgactaaaaataccatatattacagaggttaatctgatgattcgcaagaatcgtgagggtggagagctatttcagcttgtgttatcttatctatataaaatgtatgctactagcgcgcagcctaaatggtttggatcattattgcgattgttaatatgtccctggttacatatggagaaattaataggagaagcagacccggcatctacgtcggctgaaattgggtggcatatccctcgtgaacagctgatgcaagatggatggtgtggatgtgaagacggattcattccctatgttagcatacgtgcgccaagactggttatagaggagttgatggagaagaactggggccaatatcatgcccaagttattgtcactgatcagcttgtcgtaggcgaaccgcggagggtatctgctaaggctgtgatcaagggtaaccacttaccagttaagttagtttcacgatttgcatgtttcacattgacggcgaagtatgagatgaggctttcgtgcggccatagcactggacgtggagctgcatacagtgcgagactagctttccgatctgacttggcgtgatccgtgacatgcgtagtgtgacacctgctcctaggtcaatgggggtagggggcgggctaagactacgtacgcgcttcatc

SEQ ID NO: 11 - DNA, 2203 bp, unidentified
  source 1..2203: mol_type = other DNA; note = Reoviridae
gctaatctgctgaccgttactctgcaaagatggggaacgcttcctctatcgttcagacgatcaacgtcactggagatggcaatgtatttaaaccatcagctgaaacttcatctaccgctgtaccatcgttaagcttatcacctggaatgctgaatcccggaggggtaccatggattgctgttggagatgagacatctgtgacttcaccaggcgcattacgtcgaatgacgtcaaaggacatcccggaaacggcaataatcaacacagacaattcatcaggcgccgtgccaagcgaatcagccttggtgccctacatcgatgagccgctggtagtggttacagagcatgctattaccaacttcaccaaagctgagatggcacttgaawtcaatcgtgagttccttgacaagatgcgtgtgctgtcagtgtcaccaaaatattcggatcttctgacctatgttgactgctacgtcggtgtgtctgctcgtcaggctttaaacaattttcagaaacaagtgcctgtgattacacctactaggcagacgatgtatgtcgactcgatacaagcggccttgaaagctttagaaaagtgggagattgatctgagagtggctcaaacgttgctgcctacgaacgttccgattggagaagtctcttgtccaatgcagtcggtagtgaaactgctggatgatcagctgccagatgacagcctgatacggaggtatcccaaggaagccgccgtcgctttggctaaacgaaacgggggaatacaatggatggacgtatcagaaggcaccgtgatgaacgaggctgtcaacgctgttgcagctagtgcactggcaccttcagcatcagccccacccttagaagagaagtcaaagttaaccgaacaagcgatggatctcgtgaccgcggctgagcctgagataattgcctcactcgygccagttcccgcacccgtgtttgccataycacctaaaccagcagattataatgtgcgtactctgaggatcgacgaggccacttggctgcgaatgattccaaaatcaatgaacacaccttttcaaatccaggtgactgataacacaggaactaattggcatctcaatttgaggggggggactcgtgtagtgaatctggaccaaatcgctccgatgcggtttgyattagatctagggggaaagagttataaagagacgagctgggatccaaacggcaagaaggtcggattcatcgtttttcaatcgaagataccattcgaactttggactgctgcttcacagatcggtcaagccacggtggttaactatgtccaactatacgctgaagacagctcatttaccgcgcagtctatcattgctactacctctttggcttataactatgagcctgagcagttgaataagactgaccctgagatgaattattatcttttggcgacctttatagactcagccgctataacgccaacgaatatgacacagcctgatgtttgggatgccttgctgacgatgtccccactatcagctggcgaggtgacagtgaagggtgcggtagtgagtgaagtagtccctgcagacttgataggtagctacactccagaatccctaaacgcctcacttccgaatgatgctgctagatgcatgatcgatagagcttcgaagatagccgaagcaatcaagattgatgatgatgctggaccagatgaatattccccaaactctgtaccaattcaaggtcagcttgctatctcgcaactcgaaactggatatggtgtgcgaatattcaaccctaaagggatcctttctaaaattgcatctagggcaatgcaggctttcattggtgacccgagcacaatcatcacgcaggcggcgccagtgttatcagacaagaataattggattgcattggcacagggagtgaaaactagtctgcgtactaaaagtctatcagcgggagtgaagactgcagtgagtaagctgagctcatctgagtctatccagaattggactcaaggattcttggataaagtgtcagcgcattttccagcaccaaagcccgattgtccgactagcggagatagtggtgaatcgtctaatcgccgagtgaagcgcgactcatacgcaggagtggtcaaacgtgggtacacacgttaggccgctcgccctggtgacgcggggttaagggatgcaggcaaatcatc

SEQ ID NO: 12 - DNA, 2241 bp, unidentified
  source 1..2241: mol_type = other DNA; note = Reoviridae
gctaaagtgaccgtggtcatggcttcattcaagggattctccgccaacactgttccagtttctaaggccaagcgtgacatatcatctcttgccgctactcctggacttcgttcacaatccttcactccgtctgtggatatgtctcaatcgcgtgaattcctcacaaaggcaattgagcaagggtccatgtctataccttatcagcatgtgaatgtaccgaaagttgatcgtaaagttgttagcctggtagtgcgacctttctcttcaggtgctttctctatctctggagtgatttcgccagcccatgcctatctactagagtgtctaccccagcttgagcaggcgatggcttttgttgcttcacctgagtctttccaggcttccgacgtcgcgaagcgctttgccataaagccaggtatgagcctccaggatgccatcactgcctttattaactttgtgtccgcgatgctgaaaatgacggtgactcgtcaaaactttgacgttattgtggctgagatcgagaggcttgcttcaaccagcgtgtccgtcaggactgaagaagcgaaggttgctgatgaggagctaatgctattcgggttagatcatagagggccacagcagctggatgtttctgacgctaaagggataatgaaggctgctgatattcagacaactcatgatgtccatttggcaccaggcgttggtaatattgatcctgaaatctataacgaggggcggttcatgttcatgcagcacaagccacttgcggcggatcaatcgtatttcaccttggagactgcggattatttcaagatttatccaacatacgatgaacatgatggcaggatggctgaccaaaagcagtcgggattgatactgtgtactaaggacgaggtattggctgagcaaactatatttaaactggacgcccctgatgacaagactgttcatctgttggatcgcgatgacgaccacgttgttgccagatttactaaggtatttatagaggacgtggctcccgggcatcatgctgctcaaagatcgggacaacgctctgtgcttgatgacctatatgcgaatacgcaagtgatttccattacttctgctgctttaaagtgggtggtcaagcacggcgtatctgatggaatcgtgaacaggaagaatgtcaaagtgtgtgttggttttgaccccctgtacaccttgtctacacataacggggtgtccttatgtgccctgctgatggacgaaaaactctctgtgctgaacagtgcgtgtcgtatgacgttacgctcactcatgaagaccggacgcgacgttgatgcacacagagcttttcagcgagtcctctctcaaggatacacatcgctaatgtgctactatcatccttcacggaagttggcatatggtgaggtgctctttctagaacgatccaatgacgtgacagatgggatcaagcttcagttggacgcatctagacagtgtcatgaatgtcctgtgttgcagcagaaagtggttgagttagagaaacagattattatgcagaagtcaatccagtcagaccctaccccagtggcgctgcaaccattgttgtctcagttgcgtgagttgtctagtgaagttactaggctacagatggagttgagtcgagctcagtccctgaatgctcagttggaggcggatgtcaagtcagctcaatcatgtagcttggatatgtatctgagacaccacacttgcattaatggtcatgctaaagaagatgaattgcttgacgctgtgcgtgtcgcgccggatgtgaggagagaaatcatggaaaagaggagtgaagtgagacaaggttggtgcgaacgtatttctaaggaagcagctgccaaatgtcaaactgttattgatgacctgactttgatgaatggaaagcaagcacaagagataacagaattacgtgattcggctgaaaaatatgagaaacagattgcagagctggtgagtaccatcacccaaaaccagataacgtatcagcaagagctacaagccttggtagcgaaaaatgtggaattggacgcgttgaatcagcgtcaggctaagtctttgcgtattactccctctcttctatcagccactcctabcgattcagytgmtggtgttgctgacttaattgatttctctgttccaactgatgagttgtaaataatccgtgatgcagtgttgccctaatcccttaagccttcccgacccccattcatc

SEQ ID NO: 13 - DNA, 1331 bp, unidentified
  source 1..1331: mol_type = other DNA; note = Reoviridae
gctattcgctggtcagttatggctcgcgctgcgttcctattcaagactgttgggtttggtggtctgcaaaatgtgccaattaacgacgaactatcttcacatctactccgagctggtaattcaccatggcagttaacacagtttttagactggataagccttgggaggggtttagctacatcggctctcgttccgacggctgggtcaagatactatcaaatgagttgccttctaagtggcactctccagattccgttccgtcctaaccaccgatggggagacattaggttcttacgcttagtgtggtcagctcctactctcgatggattagtcgtagctccaccacaagttttggctcagcccgctttgcaagcacaggcagatcgagtgtacgactgcgatgattatccatttctagcgcgtgatccaagattcaaacatcgggtgtatcagcaattgagtgctgtaactctacttaacttgacaggttttggcccgatttcctacgttcgagtggatgaagatatgtggagtggagatgtgaaccagcttctcatgaactatttcgggcacacgtttgcagagattgcatacacattgtgtcaagcctcggctaataggccttgggaatatgacggtacatatgctaggatgactcagattgtgttatccttgttctggctatcgtatgtcggtgtaattcatcagcagaatacgtatcggacattctattttcagtgtaatcggcgaggtgacgccgctgaggtgtggattctttcttgttcgttgaaccattccgcacaaattagaccgggtaatcgtagcttattcgttatgccaactagcccagattggaacatggacgtcaatttgatcctgagttcaacgttgacggggtgtttgtgttcgggttcacagctgccactgattgacaataattcagtacctgcagtgtcgcgtaacatccatggctggactggtagagctggtaaccaattgcatgggttccaggtgagacgaatggtgactgaattttgtgacaggttgagacgcgatggtgtcatgacccaagctcagcagaatcaagttgaagcgttggcagatcagactcaacagtttaagagggacaagctcgaaacgtgggcgagagaagacgatcaatataatcaggctcatcccaactccacaatgttccgtacgaaaccatttacgaatgcgcaatggggacgaggtaatacgggggcgactagtgccgcgattgcagcccttatctgatcgtcttggagtgagggggtccccccacacccctcacgactgaccacacattcatc

SEQ ID NO: 14 - DNA, 1198 bp, unidentified
  source 1..1198: mol_type = other DNA; note = Reoviridae
gctaaagtcacgcctgtcgtcgtcactatggcttcctcactcagagctgcgatctccaagatcaagagggatgacgtcggtcagcaagtttgtcctaattatgtcatgctgcggtcctctgtcacaacaaaggtggtacgaaatgtggttgagtatcaaattcgtacgggcggattcttttcgtgcttagctatgctaaggccactccagtacgctaagcgtgagcgtttgcttggtcagaggaatctggaacgtatatcgactagggatatccttcagactcgtgatttacactcactatgtatgccaactcctgatgcgccaatgtctaatcatcaagcatccaccatgagagagctgatttgcagttacttcaaggtcgatcatgcggatgggttgaaatatatacccatggatgagagatactctccgtcatcacttgccagattgtttaccatgggcatggctgggctgcacattaccactgagccatcttataagcgtgttccgattatgcacttagctgcggacttggactgtatgacgctggctctaccttacatgattacgcttgatggtgatactgtggttcctgtcgctccaacactgtcagcggaacagcttctggacgacggactcaaaggattagcatgcatggatatctcctatggatgtgaggtggacgcgaatagccggccggctggtgatcagagtatggactcttcacgctgcatcaacgagttgtattgcgaggagacagcagaagccatctgtgtgcttaagacatgccttgtgttaaattgcatgcagtttaaacttgagatggatgacctagcacataacgctgctgagctggacaagatacagatgatgatacccttcagtgagcgtgtttttaggatggcctcgtcctttgcgactattgatgcccagtgttttaggttttgcgtgatgatgaaggataaaaatctgaaaatagatatgcgtgaaacgacgagactgtggactcgttcagcatcagatgattctgtggccacgtcatctttaagtatttccctggaccggggtcgatgggtggcggctgacgccagtgatgctagactgctggtttttccgattcgcgtgtaatgggtgagtgagctgatgtggtcgccaagacatgtgccggtgtcttggtggtgggtgacgcctaatcatc

SEQ ID NO: 15 - DNA, 1196 bp, unidentified
  source 1..1196: mol_type = other DNA; note = Reoviridae
gctatttttgcctcttcccagacgttgtcgcaatggaggtgtgcttgcccaacggtcatcaggtcgtggacttgattaacaacgcttttgaaggtcgtgtatcaatctacagcgcgcaagagggatgggacaaaacaatctcagcacagccagatatgatggtatgtggtggcgccgtcgtttgcatgcattgtctaggtgttgttggatctctacaacgcaagctgaagcatttgcctcaccatagatgtaatcaacagatccgtcatcaggattacgtcgatgtacagttcgcagaccgtgttactgctcactggaagcggggtatgctgtccttcgttgcgcagatgcacgagatgatgaatgacgtgtcgccagatgacctggatcgtgtgcgtactgagggaggttcactagtggagctgaacyggcttcaggttgacccaaattcaatgtttagatcaatacactcaagttggacagatcctttgcaggtggtggacgaccttgacactaagctggatcagtactggacagccttaaacctgatgatcgactcatccgacttgatacccaactttatgatgagagacccatcacacgcgttcaatggtgtgaaactggrgggagatgctcgtcaaacccaattctccaggacttttgattcgagatcgagtttggaatggggtgtgatggtttatgattactctgagctggagcatgatccatcgaagggccgtgcttacagaaaggaattggtgacgccagctcgagatttcggtcactttggattatcccattattctagggcgactaccccaatccttggaaagatgccggccgtattctcaggaatgttgactgggaactgtaaaatgtatccattcattaaaggaacggctaagctgaagacagtgcgcaagctagtggaggcagtcaatcatgcttggggtgtcgagaagattagatatgctcttgggccaggtggcatgacgggatggtacaataggactatgcaacaggcccccattgtgctaactcctgctgctctcacaatgttcccagataccatcaagtttggggatttgaattrtccagtgaygattggcgatccgatgattcttggctaaacacccccatcttcacagcgccgggcttgaccaacctggtgtgacgtgggacaggcttcattcatc

SEQ ID NO: 16 - DNA, 30 bp, synthetic construct
  source 1..30: mol_type = other DNA
gagcagaagttgatttcagaggaagatctg

SEQ ID NO: 17 - DNA, 24 bp, synthetic construct
  source 1..24: mol_type = other DNA
gattataaggatgacgacgataag

SEQ ID NO: 18 - DNA, 102 bp, synthetic construct
  source 1..102: mol_type = other DNA
agtattataaacttcgagaagctgggtgggggaggaagttcaatcattaattttgaaaaacttggaggaggtggatcttccataattaatttcgaaaagctg

SEQ ID NO: 19 - DNA, 693 bp, synthetic construct
  source 1..693: mol_type = other DNA
caaagttacgtggcggccgccagggtacaacctacggaatctatcgtacgcttcccgaatatcactaacttgtgtccgttcggcgaggtttttaatgcgaccaggtttgcttccgtgtacgcctggaacaggaaacggatctccaattgtgtcgccgattactccgtcttgtataattcagcatctttcagcacgtttaaatgttacggagtttcccccacaaaattgaatgacctttgctttacgaacgtctacgcggattcatttgtaatccggggggacgaagttaggcaaattgcgccagggcagactggcaagatagctgactataattataaattgccggatgactttacgggctgtgtgattgcttggaactcaaataatctggactcaaaggtagggggaaattataactacctttacaggctgttccggaagagtaatctgaagccattcgaaagagatataagtacagagatctaccaagctggaagcaccccctgcaatggtgttgaaggattcaattgttatttcccattgcaatcctatggttttcaaccgacgaatggggtgggataccaaccatatcgagttgtggttctcagtttcgagttgcttcatgctcctgcgacagtatgtggaccaaaaaaatctactaatctggtgaagaataaatgcgtcaatttttaa

SEQ ID NO: 20 - protein, 194 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 21 - protein, 194 aa, synthetic construct; identical to SEQ ID NO: 20

SEQ ID NO: 22 - DNA, 636 bp, synthetic construct
  source 1..636: mol_type = other DNA
aatatcactaacttgtgtccgttcggcgaggtttttaatgcgaccaggtttgcttccgtgtacgcctggaacaggaaacggatctccaattgtgtcgccgattactccgtcttgtataattcagcatctttcagcacgtttaaatgttacggagtttcccccacaaaattgaatgacctttgctttacgaacgtctacgcggattcatttgtaatccggggggacgaagttaggcaaattgcgccagggcagactggcaagatagctgactataattataaattgccggatgactttacgggctgtgtgattgcttggaactcaaataatctggactcaaaggtagggggaaattataactacctttacaggctgttccggaagagtaatctgaagccattcgaaagagatataagtacagagatctaccaagctggaagcaccccctgcaatggtgttgaaggattcaattgttatttcccattgcaatcctatggttttcaaccgacgaatggggtgggataccaaccatatcgagttgtggttctcagtttcgagttgcttcatgctcctgcgacagtatgtggaccaaaaaaatctactaatctggtgaagaataaatgcgtcaatttttaa

SEQ ID NO: 23 - protein, 8 aa, synthetic construct
SIINFEKL

SEQ ID NO: 24 - protein, 62 aa, synthetic construct
GGGGSGGGGSEQKLISEEDLSIINFEKLGGGGSSIINFEKLGGGGSSIINFEKLDYKDDDDK

SEQ ID NO: 25 - DNA, 189 bp, synthetic construct
  source 1..189: mol_type = other DNA
ggaggtggaggctcaggtggcggaggttctgagcagaagttgatttcagaggaagatctgagtattataaacttcgagaagctgggtgggggaggaagttcaatcattaattttgaaaaacttggaggaggtggatcttccataattaattttgaaaagctggattataaggatgacgacgataagtga

SEQ ID NO: 26 - protein, 17 aa, synthetic construct
ISQAVHAAHAEINEAGR

SEQ ID NO: 27 - protein, 45 aa, synthetic construct
GGGGSGGGGSEQKLISEEDLISQAVHAAHAEINEAGRDYKDDDDK

SEQ ID NO: 28 - DNA, 138 bp, synthetic construct
  source 1..138: mol_type = other DNA
ggaggtggaggctcaggtggcggaggttctgagcagaagttgatttcagaggaagatctgatttcacaggctgtgcatgctgcacatgctgaaattaatgaggctggacgtgattataaggatgacgacgataagtga

SEQ ID NO: 29 - protein, 9 aa, synthetic construct
ASMTNMELM

SEQ ID NO: 30 - protein, 65 aa, synthetic construct
GGGGSGGGGSEQKLISEEDLASMTNMELMGGGGSASMTNMELMGGGGSASMTNMELMDYKDDDDK

SEQ ID NO: 31 - DNA, 198 bp, synthetic construct
  source 1..198: mol_type = other DNA
ggaggtggaggctcaggtggcggaggttctgagcagaagttgatttcagaggaagatctggctagcatgacgaacatggagctaatgggagggggcggaagtgcttctatgactaatatggaactgatgggaggtggaggttctgcttcaatgaccaacatggaacttatggattataaggatgacgacgataagtga

SEQ ID NO: 32 - protein, 8 aa, synthetic construct
KILTFDRL

SEQ ID NO: 33 - protein, 62 aa, synthetic construct
GGGGSGGGGSEQKLISEEDLKILTFDRLGGGGSKILTFDRLGGGGSKILTFDRLDYKDDDDK

SEQ ID NO: 34 - DNA, 189 bp, synthetic construct
  source 1..189: mol_type = other DNA
ggaggtggaggctcaggtggcggaggttctgagcagaagttgatttcagaggaagatctgaagattctgacgtttgatcgtctggggggtggcggatctaagatattgacgttcgatcgactgggaggaggtggctctaagatcctaacattcgaccgtctagattataaggatgacgacgataagtga

SEQ ID NO: 35 - protein, 8 aa, synthetic construct
KSPWFTTL

SEQ ID NO: 36 - protein, 62 aa, synthetic construct
GGGGSGGGGSEQKLISEEDLKSPWFTTLGGGGSKSPWFTTLGGGGSKSPWFTTLDYKDDDDK

SEQ ID NO: 37 - DNA, 189 bp, synthetic construct
  source 1..189: mol_type = other DNA
ggaggtggaggctcaggtggcggaggttctgagcagaagttgatttcagaggaagatctgaagtcaccttggtttactactctcggtgggggaggaagtaaatcgccatggtttactacattgggaggaggtgggtctaagtcaccatggttcacgacattggattataaggatgacgacgataagtga

SEQ ID NO: 38 - protein, 18 aa, synthetic construct
PSKPSKRSFIEDLLFNKV

SEQ ID NO: 39 - protein, 46 aa, synthetic construct
GGGGSGGGGSEQKLISEEDLPSKPSKRSFIEDLLFNKVDYKDDDDK

SEQ ID NO: 40 - DNA, 141 bp, synthetic construct
  source 1..141: mol_type = other DNA
ggaggtggaggctcaggtggcggaggttctgagcagaagttgatttcagaggaagatctgcctagtaagccttctaagcgcagcttcatagaagatttgttgttcaataaggtagattataaggatgacgacgataagtga

SEQ ID NO: 41 - protein, 18 aa, synthetic construct
PSKPSKRSFIEDLLFNKV

SEQ ID NO: 42 - protein, 69 aa, synthetic construct
GGGGSGGGGSEQKLISEEDLPSKPSKRSFIEDLLFNKVGGGGSPSKPSKRSFIEDLLFNKVDYKDDDDK

SEQ ID NO: 43 - DNA, 210 bp, synthetic construct
  source 1..210: mol_type = other DNA
ggaggtggaggctcaggtggcggaggttctgagcagaagttgatttcagaggaagatctgcctagtaagccttctaagcgcagcttcatagaagatttgttgttcaataaggtaggtgggggaggaagtccatcgaagccaagtaagcgtagtttcattgaggacctcctcttcaacaaggttgattataaggatgacgacgataagtga

(19) United States
(12) Patent Application Publication
(10) Pub. No.: US 2025/0154204 A1
(43) Pub. Date: May 15, 2025

(54) NOVEL REOVIRUS-BASED VACCINE PLATFORM AND USE THEREOF

(71) Applicant: VIROCURE, INC., Seoul (KR)

(72) Inventors: Haeng Jun YOO, Seoul (KR); Tommy Michel ALAIN, Ottawa (CA); Sang Kyoung HAN, Seoul (KR); Yeon Sook LEE, Gyeonggi-do (KR); Xiao XIANG, Ottawa (CA)

(21) Appl. No.: 18/694,516

(22) PCT Filed: Sep. 26, 2022
(86) PCT No.: PCT/KR2022/014373

§ 371 Date: Mar. 22, 2024

(30) Foreign Application Priority Data
Sep. 24, 2021 (KR) 10-2021-0126694
Sep. 26, 2022 (KR) 10-2022-0121618

(51) Int. Cl. C07K 14/195; A61K 39/00; A61K 39/12; A61K 39/13; A61K 39/145; A61K 39/155; A61K 39/165; A61K 39/205; A61K 39/215; A61K 39/235; A61K 39/29; A61P 37/04; C07K 14/005; C12N 15/86

(52) U.S. Cl. CPC C07K 14/195; A61K 39/001104; A61K 39/001109; A61K 39/001112; A61K 39/001113; A61K 39/001119; A61K 39/001129; A61K 39/001153; A61K 39/001168; A61K 39/001182; A61K 39/001188; A61K 39/12; A61K 39/13; A61K 39/145; A61K 39/155; A61K 39/165; A61K 39/205; A61K 39/215; A61K 39/235; A61K 39/29; A61K 39/292; A61P 37/04; C07K 14/005; C12N 15/86; A61K 2039/5256; C07K 2319/00; C12N 2720/12043; C12N 2770/20022; C12N 2770/20034; C12N 2770/20071

(57) ABSTRACT

The present invention relates to a novel reovirus-based vaccine platform, and confirmed that a part of the S1 gene of reovirus can be replaced with various exogenous epitope-encoding genes, and a recombinant reovirus manufactured according to the present invention not only can infect target cells and induce the expression of the epitope, but also can effectively prevent and treat diseases related to the epitope by activating the immune function of immune cells against the epitope. When using the reovirus-based vaccine platform of the present invention, vaccines containing various epitopes can be manufactured through relatively simple genetic manipulation technology, and can be administered in various ways including oral administration, so it can be utilized for the prevention and treatment of various infectious diseases including SARS-CoV-2 virus infection, and cancer.

### CROSS-REFERENCE TO RELATED APPLICATIONS

This application is a 371 of PCT/KR2022/014373 on Sep. 6, 2022, which claims the benefit of priority from Korean Patent Application No. 10-2021-0126694 filed on Sep. 24, 2021 and Korean Patent Application No. 10-2022-0121618 filed on Sep. 26, 2022, the contents of each of which are incorporated herein by reference in its entirety.

### TECHNICAL FIELD

The present invention relates to a novel reovirus-based vaccine platform and use thereof.

### SEQUENCE LISTING

This application contains references to amino acid sequences and/or nucleic acid sequences which have been submitted concurrently herewith as the sequence listing XML file entitled “000010usnp_SequenceListing_Revised.xml”, file size 68,000 bytes (B), created on 27 Sep. 2024. The aforementioned sequence listing is hereby incorporated by reference in its entirety pursuant to 37 C.F.R. § 1.52(e)(5).

### BACKGROUND ART

Viruses are small non-cellular organisms made up of genetic material and proteins. There are different types of viruses. For example, viruses may be DNA viruses that replicate within the host's nucleus, or RNA viruses that replicate within the cytoplasm of a cell. Viruses can be double-stranded or single-stranded. Moreover, single-stranded RNA viruses can be either positive (+, sense) strands or negative (−) strands. These different types of viruses cause a variety of viral infections.

Today, the most important protective measure to combat viral infections and limit their spread is vaccination. Modern vaccines principally induce the formation of antibodies against surface viral antigens. Vaccine effectiveness is directly dependent on the degree of match between the antigenic structure of a virus contained in the vaccine and strains circulating in the population. The surface proteins of most viruses undergo constant antigenic variation (antigenic drift), necessitating continuous updating of vaccine strain composition. The development of highly immunogenic and safe vaccines that induce broad-spectrum immune responses is one of the major challenges currently encountered in efficient cancer or infectious disease prevention. The development of a vaccine is necessary to prevent the spread and continued infection of global pandemic diseases such as the recently spread coronavirus disease 2019 (COVID-19) caused by SARS-CoV-2. Accordingly, many virus-based vaccines are currently being developed worldwide using platforms such as vesicular stomatitis virus (VSV), measles virus (MeV), adenovirus (Ad), baculovirus, etc.

Various vaccines are being developed to prevent tumors as well as infectious diseases caused by viruses, bacteria, and fungi. In particular, research is being actively conducted on delivery vehicles that can carry various antigens and stably deliver attenuated antigens into the body. Various polymer-based nanoparticles, exosomes, peptides, etc. have been used as the delivery vehicles, but they have problems such as stability and toxicity in the body. In particular, there is a limitation in that stable antigen delivery is impossible. Accordingly, attempts have been made to manufacture a viral vector-based vaccine based on the replication potential of a virus and its ability to induce gene expression, but a viral-based vector that can stably express exogenous antigens without causing side effects in the body has not yet been discovered.

Meanwhile, oncolytic viruses are viruses that can replicate on their own and selectively infect, proliferate, and kill only cancer cells, not normal cells, and are emerging as a fourth-generation anticancer drug that will change the anticancer drug market in the future. The destruction of tumor cells by oncolytic viruses has the advantage of re-inducing re-infection of surrounding tumor cells and repeating this phenomenon, amplifying the anticancer effect. In particular, in addition to directly attacking cancer cells, oncolytic viruses also have the function of inhibiting the formation of new blood vessels through infection within tumor vascular endothelial cells.

A representative oncolytic virus is a reovirus. reovirus is a virus characterized by no envelope and infects cells through an oral-fecal route. The genome of the reovirus has 10 segmented dsRNAs encoding 8 structural and 3 non-structural proteins, and mammalian reovirus exists in four serotypes: T1L (Lang), T2J (Jones), T3D (Dearing), T3A (Abney), and T4N (Ndelle). It is known that reovirus can efficiently infect and lyse cancer cells. In addition, clinical trials were conducted on intratumoral or intravenous administration of reovirus, so high treatment safety and anticancer efficacy thereof were confirmed.

There have been attempts to use the oncolytic virus as a vaccine platform for various diseases as well as cancer based on its repeatability and anticancer efficacy, but the entry barrier to oncolytic virus immunotherapy is high due to the difficulty in development and production technology.

### DISCLOSURE

### Technical Problem

Therefore, the present invention has been made in view of the above problems and completed by confirming that a mutant S1 gene of reovirus can fuse with an exogenous epitope-encoding gene, and accordingly, it can be used as a vaccine platform for the prevention and treatment of various diseases.

Therefore, it is one object of the present invention to provide a recombinant vector containing the mutant S1 gene of reovirus and the exogenous epitope-encoding gene.

It is another object of the present invention to provide a mutant S1 protein fused with an exogenous epitope-coding gene.

It is still another object of the present invention to provide a cell into the recombinant vector according to the present invention has been introduced.

It is still another object of the present invention to provide a recombinant reovirus including the mutant S1 gene and an exogenous epitope-encoding gene.

It is still another object of the present invention to provide a vaccine composition comprising the recombinant vector according to the present invention, a cell into which the vector has been introduced, or reovirus expressed from the recombinant vector as an active ingredient.

It is still another object of the present invention to provide a pharmaceutical composition for preventing or treating viral infections, the pharmaceutical composition comprising a recombinant vector as an active ingredient, the recombinant vector including the mutant S1 gene of reovirus and a viral epitope-encoding gene.

It is yet another object of the present invention to provide a pharmaceutical composition for preventing or treating cancer, the pharmaceutical composition comprising a recombinant vector as an active ingredient, the recombinant vector including the mutant S1 gene of reovirus and an epitope-encoding gene of a tumor antigen.

It will be understood that technical problems of the present invention are not limited to the aforementioned problem and other technical problems not referred to herein will be clearly understood by those skilled in the art, to which the present invention pertains, from the description below.

### Technical Solution

In accordance with an aspect of the present invention, the above and other objects can be accomplished by the provision of a recombinant vector, comprising a mutant S1 gene of reovirus and an exogenous epitope-encoding gene, wherein the mutant S1 gene has a stop codon at a 251st codon from a start codon thereof.

In an embodiment of the present invention, the mutant S1 gene may encode a polypeptide comprising an amino acid sequence of SEQ ID NO: 1 or 2, without being limited thereto.

In another embodiment of the present invention, the mutant S1 gene may comprise one polynucleotide sequence selected from the group consisting of:
(a) a polynucleotide sequence of SEQ ID NO: 4 or 5; and
(b) a polynucleotide sequence of SEQ ID NO: 6 in which a 763rd nucleotide from a 5′ terminal is substituted with T.

In another embodiment of the present invention, the exogenous epitope-encoding gene may be located downstream of the mutant S1 gene, without being limited thereto.

In another embodiment of the present invention, the mutant S1 gene and the exogenous epitope-encoding gene may be expressed together to produce a fusion protein, without being limited thereto.

In another embodiment of the present invention, the exogenous epitope may be one or more selected from the group consisting of a viral antigen, a bacterial antigen, a fungal antigen, an allergen, and a tumor antigen.

In another embodiment of the present invention, the virus may be one or more selected from the group consisting of SARS-CoV-2 virus, norovirus, influenza virus, Ebola virus, human papillomavirus, hepatitis B virus, hepatitis C virus, hepatitis D virus, Marburg virus, human parainfluenza virus 1, measles virus, mumps virus, rabies virus, human immunodeficiency virus, dengue virus, poliovirus, cytomegalovirus, dengue virus, yellow fever virus, adenovirus, Japanese encephalitis virus, smallpox virus and Zika virus, without being limited thereto.

In another embodiment of the present invention, the tumor antigen may be one or more selected from the group consisting of ovalbumin, CD19, NY-ESO-1, EGFR, TAG72, IL13Rα2 (interleukin 13 receptor alpha-2 subunit), CD52, CD33, CD20, TSLPR, CD22, CD30, GD3, CD171, NCAM (neural cell adhesion molecule), FBP (folate binding protein), Le(Y) (Lewis-Y antigen), PSCA (prostate stem cell antigen), PSMA (prostate-specific membrane antigen), CEA (carcinoembryonic antigen), HER2 (human epidermal growth factor receptor 2), mesothelin, CD44v6 (hyaluronate receptor variant 6), B7-H3, glypican-3, ROR1 (receptor tyrosine kinase like orphan receptor 1), survivin, FOLR1 (folate receptor), WT1 (Wilm's tumor antigen), VEGFR2 (vascular endothelial growth factor 2), tumor virus antigen, TP53, and KRAS, without being limited thereto.

In another embodiment of the present invention, the recombinant vector may comprise one or more genes selected from the group consisting of L1, L2, L3, M1, M2, M3, S2, S3, and S4, without being limited thereto.

In accordance with another aspect of the present invention, there is provided a mutant Sigma 1 protein expressed from the recombinant vector and fused with an exogenous epitope.

In accordance with another aspect of the present invention, there is provided a cell into which the recombinant vector has been introduced.

In an embodiment of the present invention, a vector comprising one or more genes selected from the group consisting of L1, L2, L3, M1, M2, M3, S2, S3, and S4 of reovirus may be further introduced into the cell, without being limited thereto.

In accordance with another aspect of the present invention, there is provided a recombinant reovirus, comprising a mutant S1 gene and an exogenous epitope-encoding gene, wherein the mutant S1 gene has a stop codon at a 251st codon from a start codon thereof.

In an embodiment of the present invention, the reovirus may express the exogenous epitope, without being limited thereto. Preferably, the exogenous epitope may be expressed as an outer capsid protein.

In another embodiment of the present invention, the recombinant reovirus may be produced from a cell into which the recombinant vector according to the present invention has been introduced, without being limited thereto.

In accordance with another aspect of the present invention, there is provided a vaccine composition comprising the recombinant vector according to the present invention, a cell into which the recombinant vector has been introduced, or recombinant reovirus produced from the cell as an active ingredient. The vaccine may be a vaccine for preventing infectious diseases and/or cancer. Preferably, the infectious diseases and/or cancer may be diseases and/or cancer related to the exogenous epitope.

In accordance with another aspect of the present invention, there is provided use of the recombinant vector, a cell into which the recombinant vector has been introduced, or recombinant reovirus produced from the cell for manufacture of a vaccine for preventing infectious diseases and/or cancer.

In accordance with another aspect of the present invention, there is provided a pharmaceutical composition for preventing or treating viral infections, the pharmaceutical composition comprising: a recombinant vector including a mutant S1 gene of reovirus and a viral epitope-encoding gene; a cell into which the recombinant vector has been introduced; or recombinant reovirus produced from the cell as an active ingredient, wherein the mutant S1 gene has a stop codon at a 251st codon from a start codon thereof.

In accordance with another aspect of the present invention, there is provided a food composition for preventing or alleviating viral infections, the food composition comprising: a recombinant vector including a mutant S1 gene of reovirus and a viral epitope-encoding gene; a cell into which the recombinant vector has been introduced; or recombinant reovirus produced from the cell as an active ingredient, wherein the mutant S1 gene has a stop codon at a 251st codon from a start codon thereof. The food composition includes a healthy functional food composition.

In accordance with another aspect of the present invention, there is provided a method of preventing or treating viral infections, the method comprising administering the recombinant vector, a cell into which the recombinant vector has been introduced, or recombinant reovirus produced from the cell to an individual in need thereof.

In accordance with another aspect of the present invention, there is provided use of the recombinant vector, a cell into which the recombinant vector has been introduced, or recombinant reovirus produced from the cell to manufacture a medicine for preventing or treating viral infections.

In accordance with another aspect of the present invention, there is provided use of the recombinant vector, a cell into which the recombinant vector has been introduced, or recombinant reovirus produced from the cell for preventing or treating viral infections.

In an embodiment of the present invention, the virus may be one or more selected from the group consisting of SARS-CoV-2 virus, norovirus, influenza virus, Ebola virus, human papillomavirus, hepatitis B virus, hepatitis C virus, hepatitis D virus, Marburg virus, human parainfluenza virus 1, measles virus, mumps virus, rabies virus, human immunodeficiency virus, dengue virus, poliovirus, cytomegalovirus, dengue virus, yellow fever virus, adenovirus, Japanese encephalitis virus, smallpox virus and Zika virus, without being limited thereto.

In accordance with another aspect of the present invention, there is provided a pharmaceutical composition for preventing or treating cancer, the pharmaceutical composition including a recombinant vector comprising a mutant S1 gene of reovirus and an epitope-encoding gene of a tumor antigen; a cell into which the recombinant vector has been introduced; or recombinant reovirus produced from the cell, wherein the mutant S1 gene has a stop codon at a 251st codon from a start codon thereof, as an active ingredient.

In accordance with another aspect of the present invention, there is provided a food composition for preventing or alleviating cancer, the food composition comprising a recombinant vector including a mutant S1 gene of reovirus and an epitope-encoding gene of a tumor antigen; a cell into which the recombinant vector has been introduced; or recombinant reovirus produced from the cell as an active ingredient, wherein the mutant S1 gene has a stop codon at a 251st codon from a start codon thereof. The food composition includes a healthy functional food composition.

In accordance with another aspect of the present invention, there is provided a method of preventing or treating cancer, the method comprising administering the recombinant vector, a cell into which the recombinant vector has been introduced, or recombinant reovirus produced from the cell to an individual in need thereof.

In accordance with another aspect of the present invention, there is provided use of the recombinant vector, a cell into which the recombinant vector has been introduced, or recombinant reovirus produced from the cell for manufacture of a medicine for preventing or treating cancer.

In accordance with yet another aspect of the present invention, there is provided use of the recombinant vector, a cell into which the recombinant vector has been introduced, or recombinant reovirus produced from the cell for preventing or treating cancer.

In an embodiment of the present invention, the tumor antigen may be one or more selected from the group consisting of ovalbumin, CD19, NY-ESO-1, EGFR, TAG72, IL13Rα2 (interleukin 13 receptor alpha-2 subunit), CD52, CD33, CD20, TSLPR, CD22, CD30, GD3, CD171, NCAM (neural cell adhesion molecule), FBP (folate binding protein), Le(Y) (Lewis-Y antigen), PSCA (prostate stem cell antigen), PSMA (prostate-specific membrane antigen), CEA (carcinoembryonic antigen), HER2 (human epidermal growth factor receptor 2), mesothelin, CD44v6 (hyaluronate receptor variant 6), B7-H3, glypican-3, ROR1 (receptor tyrosine kinase like orphan receptor 1), survivin, FOLR1 (folate receptor), WT1 (Wilm's tumor antigen), VEGFR2 (vascular endothelial growth factor 2), tumor virus antigen, TP53, and KRAS, without being limited thereto.

In another embodiment of the present invention, the cancer may be one or more selected from the group consisting of squamous cell carcinoma, lung cancer, adenocarcinoma of lung, peritoneal cancer, skin cancer, melanoma, skin melanoma, intraocular melanoma, rectal cancer, anal cancer, esophageal cancer, small intestine cancer, endocrine cancer, parathyroid cancer, adrenal cancer, soft tissue sarcoma, urethral cancer, blood cancer, liver cancer, gastrointestinal cancer, pancreatic cancer, glioblastoma, cervical cancer, ovarian cancer, bladder cancer, liver tumor, breast cancer, colon cancer, colorectal cancer, endometrial or uterine cancer, salivary gland cancer, kidney cancer, prostate cancer, vulva cancer, thyroid cancer, head and neck cancer and brain cancer, without being limited thereto.

### Advantageous Effects

The present invention relates to a novel reovirus-based vaccine platform, and confirmed that a part of the S1 gene of reovirus can be replaced with various exogenous epitope-encoding genes, and a recombinant reovirus manufactured according to the present invention not only can infect target cells and induce the expression of the epitope, but also can effectively prevent and treat diseases related to the epitope by activating the immune function of immune cells against the epitope. When using the reovirus-based vaccine platform of the present invention, vaccines containing various epitopes can be manufactured through relatively simple genetic manipulation technology, and can be administered in various ways including oral administration, so it can be utilized for the prevention and treatment of various infectious diseases including SARS-CoV-2 virus infection, and cancer.

### BRIEF DESCRIPTION OF DRAWINGS

FIG. 1a illustrates a comparison diagram of the σ1 protein of the wild-type reovirus (left) and the mutated σ1 protein of reovirus RP116 (right).

FIG. 1b illustrates the manufacturing principle of a reovirus-based vaccine platform according to the present invention.

FIG. 1c illustrates the operating principle of the reovirus-based vaccine platform according to the present invention.

FIG. 1d illustrates the manufacturing principle of a reovirus-based vaccine according to the present invention.

FIG. 2a illustrates reovirus protein detection results obtained by Western blotting after infecting cells with a negative control group (Mock); wild-type reovirus (WT ReoV); mutant reovirus (ReoV+Q251*) wherein a STOP codon mutation is present at the 251st amino acid position of S1 gene; or recombinant reovirus (ReoV+RBD) wherein mutant S1 gene is fused with SARS-Cov-2 receptor binding domain (RBD) so as to investigate the ability of the vaccine platform according to one embodiment of the present invention to induce the production of a neutralizing antibody against the SARS-CoV-2 virus.

FIG. 2b illustrates RBD gene detection results, obtained through RT-qPCR, of cells infected with Mock, WT ReoV, ReoV+Q251*, or ReoV+RBD.

FIG. 2c illustrates the detection results of SARS-CoV-2 neutralizing antibody (NeuAb; upper) and anti-reovirus antibody (lower) in cells infected with Mock, WT ReoV, ReoV+Q251*, or ReoV+RBD.

FIG. 3 illustrate the detection results of foreign epitope RNA products present in cell lysates after manufacturing various recombinant reoviruses having a mutant S1 gene fused with T cell epitope and infecting cells with the recombinant reoviruses so as to investigate the foreign antigen expression ability of the reovirus-based vaccine platform of the present invention.

FIGS. 4a and 4b illustrate the detection results of reovirus-derived proteins (FIG. 4a) and foreign epitope RNA products (FIG. 4b) present in cell lysates after manufacturing various recombinant reoviruses having a mutant S1 gene fused with B cell epitope and infecting cells with the recombinant reoviruses so as to investigate the foreign antigen expression ability of the reovirus-based vaccine platform of the present invention.

FIG. 5 illustrates the detection results of the fluorescence signals of reovirus proteins and tag proteins after infecting mammalian cells with reovirus into which the ovalbumin epitope (OVA257-264) tagged with Myc and FLAG was introduced (scale bar=50 μm).

FIG. 6 illustrates a schematic diagram of the S1 gene of recombinant reovirus into which S21P2 or OVA257-264 epitope tagged with Myc and FLAG was introduced.

FIG. 7a is a schematic diagram illustrating the process of administering a reovirus vaccine (reovirus vaccine into which SARS-CoV-2 antigen S21P2 has been introduced) according to one embodiment to mice so as to investigate the in vivo efficacy of the reovirus-based vaccine according to the present invention.

FIG. 7b is a graph comparing the body weights of mice administered with wild-type or recombinant reovirus.

FIG. 7c is a graph comparing the inhibition ability of HEK293T cell infection by a neutralizing antibody isolated from the serum of mice administered wild-type or recombinant reovirus.

FIG. 7d illustrates the detection result of SARS-CoV-2 spike protein-specific immunoglobulin in the serum of mice administered with wild-type or recombinant reovirus.

FIG. 8a illustrates the detection result of epitopes bound to MHC-I by flow cytometry after infecting cells with various recombinant reoviruses (SIINFEKL: OVA257-264 epitope).

FIGS. 8b and 8c illustrate the measurement result of the proportion of CD8+ T cells expressing TNFα and/or INFγ by flow cytometry after infecting cells with various recombinant reoviruses (FIG. 8b) and a quantification graph thereof (FIG. 8c).

FIG. 9a illustrates RP116 reovirus σ1 protein fused with a tumor epitope.

FIG. 9b illustrates the detection result (right) of Myc and FLAG proteins tagged to reovirus protein (left) and ovalbumin epitope (OVA257-267) by Western blot after infecting cells with various reoviruses for 72 hours.

FIG. 9c illustrates the principle of inducing an immune response of the reovirus-based vaccine platform of the present invention.

FIG. 10a illustrates the process of administering reovirus to which wild-type or foreign epitope (OVA257-264) has been introduced into mice and then injecting tumor cells thereinto so as to investigate the cancer prevention effect of the reovirus-based vaccine of the present invention in vivo.

FIG. 10b illustrates the comparison result of the tumor sizes in the mice injected with tumor cells after reovirus administration. In each individual, the tumor on the left is a tumor developed from general B16F10 cells, and the tumor on the right is a tumor developed from B16F10 cells expressing ovalbumin-specific antigen.

FIG. 10c illustrates the comparison graph of the body weight of mice administered with wild-type or recombinant reovirus.

FIG. 10d illustrates a quantification result of the size of the tumor developed from general B16F10 cells after reovirus administration and the size of the tumor developed from B16F10 cells expressing the ovalbumin-specific antigen.

FIG. 11a illustrates a comparing result of the degree of tumor growth over time after administering recombinant reovirus to a melanoma mouse model (Vehicle, untreated control group; S1-Q251*, group administered with recombinant reovirus without epitope; and S1-OVA, recombinant reovirus administration group administered with ovalbumin antigen).

FIGS. 11b and 11c illustrate the results of observing (FIG. 11b) and quantifying (FIG. 11c) the level of OVA-specific T cell activation generated after administering recombinant reovirus to a melanoma mouse model.

### BEST MODE

The present invention is about a quick change-based vaccine platform using reverse genetics technology of reovirus, in which the head portion of the Sigma 1 protein (δ1 protein) of reovirus may be replaced with various foreign peptides. Accordingly, the present invention was completed by confirming that it can be used as a vaccine for the prevention or treatment of various diseases, including infectious diseases and cancer, by introducing various antigen epitopes. That is, the present inventors developed a vaccine platform based on an attenuated reovirus using reverse genetics technology and a “quick exchange” method that an epitope base sequence is inserted into a reovirus Sigma 1 protein attenuated by cutting a specific amino acid sequence position therein such that an epitope for cancer or infectious disease is fused and expressed.

Specifically, the Sigma 1 protein according to the present invention is a truncated Sigma 1 protein in which amino acids 251 to 455 from the N terminus are deleted compared to the wild-type Sigma 1 protein. The truncated Sigma 1 is encoded by the mutant S1 gene into which a stop codon has been introduced. The mutant S1 gene is characterized in that the 251st codon (i.e., ⁷⁶³CAA, encoding ²⁵¹Q) from the start codon (initiation codon) is replaced with a stop codon as the 763rd nucleotide C from the 5′ terminal is replaced with T. The present inventors confirmed that when the gene encoding the exogenous peptide is linked after the stop codon, the exogenous peptide is fused and expressed with the truncated Sigma 1, and as a result, it is possible to construct a recombinant reovirus that can infect cells and stably induce the expression of an exogenous peptide. Accordingly, the present inventors produced various recombinant reoviruses by introducing various antigen proteins, including SARS-CoV-2 epitopes and tumor antigens, downstream of the mutant S1 gene, and confirmed that the recombinant reoviruses can stably induce the expression of the introduced antigens in infected cells and activate the immune function of immune cells against the antigens.

The recombinant reovirus-based vector according to the present invention has no limitations on the type of antigen that can be introduced, so it can be used as a vaccine platform for the prevention and treatment of cancer as well as infectious diseases caused by various viruses, bacteria, and fungi. In addition, the recombinant reovirus is a safe virus that has been confirmed not to cause disease in itself, so there is no risk of unexpected side effects, and it can maintain stability even under harsh environmental conditions, so it may be stored not only at room temperature or low temperatures of 4° C., but also at temperatures as low as −20° C. Accordingly, the composition according to the present invention has the advantage of being able to be administered orally in the form of a beverage or food. In addition, since reoviruses are highly productive and have well-established production processes, large quantities of virus-based vaccines can be produced at relatively low costs.

Accordingly, the main purpose of the present invention is to provide a recombinant vector containing the mutant S1 gene of reovirus and an exogenous epitope-encoding gene.

Hereinafter, the present invention is described in detail.

The present invention relates to a recombinant vector containing the mutant S1 gene of reovirus and an exogenous epitope-encoding gene, wherein Here, the mutant S1 gene has a stop codon at the 251st codon from the start codon thereof.

A respiratory enteric orphan (REO) virus is a non-enveloped icosahedral virus with a double-stranded RNA fragment as its genome. reovirus is a non-pathogenic virome that is commonly isolated from the digestive and respiratory tracts of healthy humans. In particular, reovirus is known as an oncolytic virus that can infect and kill various tumor cells. That is, like general oncolytic viruses, reovirus has the advantage of having low side effects because it can specifically infect cancer cells and induce their death while having little effect on normal cells, and the advantage of being able to proliferate in primary infected cancer cells and then infect surrounding and distant cancer cells, causing a wide range of anticancer effects. The reovirus genome is composed of ten individual segments encoding eight structural proteins and three non-structural proteins: three large segments (L1, L2, L3), three medium segments (M1, M2, M3), and four small segments (S1, S2, S3, and S4). The outer capsid of reovirus is composed of four proteins sigma-1, sigma-3, lambda-2, and mu-1. Sigma-1 is a cell-adhesion protein of reovirus that binds to a receptor on target cells, allowing the virus to infect the cells.

As described above, wild-type reovirus has utility as an oncolytic virus. However, the wild-type reovirus has a problem in that its anti-cancer function may be weakened due to neutralizing antibodies when injected into the body, and there is a risk that the anti-cancer function of reovirus may be suppressed by the tumor microenvironment of cancer cells. Furthermore, there is still a problem that the reovirus can cause abnormalities in a host by infecting normal cells rather than cancer cells. To solve the problem of host toxicity of the wild-type reovirus, the present inventors manufactured an attenuated reovirus (AV) that expresses a truncated form of Sigma 1 protein (i.e., mutant Sigma 1 protein) due to the presence of a premature STOP codon (TAA) in the middle of the wild-type Sigma 1 protein coding gene. The attenuated reovirus is characterized by further reduced toxicity to a host compared to the wild-type reovirus. That is, in a gene (S1 gene) encoding sigma 1, an attachment protein exposed to the outside, of the capsid of the wild-type reovirus, the attenuated reovirus according to the present invention may include a mutation in which 763CAA is converted into 763TAA (stop codon) as the 763rd C, which encodes glutamine (Q) as the 251st amino acid of the protein, from the 5′ terminal, is substituted with T. In addition, the attenuated reovirus according to the present invention may be characterized in that the spherical head is cut (deleted) due to cutting (deletion) from the 251st amino acid of the wild-type Sigma 1 protein due to the immature stop codon mutation. In an embodiment of the present invention, the attenuated reovirus may be RP116.

Specifically, the Sigma 1 protein according to the present invention is a truncated Sigma 1 protein N from the terminal of which 251th to 455th amino acids have been deleted, compared to the wild-type Sigma 1 protein. In other words, the Sigma 1 protein according to the present invention is characterized by including the 1st to 250th amino acid sequences of the wild-type reovirus Sigma 1 protein, preferably consisting of the 1st to 250th amino acid sequences. The truncated Sigma 1 protein, i.e., the mutated Sigma 1 protein, is characterized by being attenuated compared to the Sigma 1 protein of the wild-type reovirus. The Sigma 1 protein of the wild-type reovirus is encoded by the S1 segment (also referred to as “S1 gene”, etc.). Accordingly, the mutant Sigma 1 protein according to the present invention may be encoded by the mutant S1 gene with a stop codon at the 251st codon from the start codon thereof.

More specifically, the mutant Sigma 1 protein according to the present invention may include the amino acid sequence of SEQ ID NO: 1 or may be represented by the amino acid sequence of SEQ ID NO: 1 or 2. Alternatively, the mutant Sigma 1 protein according to the present invention may have a deletion of the 251th to 455th amino acids of the amino acid sequence of SEQ ID NO: 3.

In addition, the mutant S1 gene according to the present invention may be characterized by having a stop codon at the 251st codon from the start codon thereof. The substitution with the stop codon may be caused by a substitution of the 763rd nucleotide from the 5′ terminal of the S1 gene with T.

More specifically, the mutant S1 gene according to the present invention may include or consist of may include or consist of one polynucleotide sequence selected from the group consisting of:
(a) the polynucleotide sequence of SEQ ID NO: 4 or 5; and
(b) the polynucleotide sequence of SEQ ID NO: 6 in which the 763rd nucleotide from the 5′ terminal is substituted with T.

SEQ ID NO: 4 is a polynucleotide consisting of 1st to 765th nucleotides among the polynucleotide sequence of SEQ ID NO: 5.

The base sequence of each gene segment of the modified reovirus according to the present invention and the amino acid sequence of each protein encoded by the base sequence are listed in the sequence list of this specification.

In the present invention, a gene (nucleic acid molecule) with a specific sequence number may include the nucleotide sequence (polynucleotide sequence) of the sequence number or may consist of the nucleotide sequence of the SEQ ID NO, and so long as the purpose and function of the modified reovirus according to the present invention are maintained, variants of the base sequence are included within the scope of the present invention. For example, a nucleic acid molecule with a base sequence indicated by a specific sequence number is a concept that includes functional equivalents of nucleic acid molecules thereof, for example, variants in which some base sequences of a nucleic acid molecule have been modified by deletion, substitution, or insertion, but can have the same functional effect as the nucleic acid molecule. Specifically, a nucleic acid molecule represented by a specific sequence number may include a base sequence having a sequence homology of at least 70%, more preferably at least 80%, even more preferably at least 90%, and most preferably at least 95% of a base sequence represented by the corresponding sequence number. For example, a polynucleotide having a sequence homology of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% is included. The “% sequence homology” for a polynucleotide is determined by comparing a comparison region with two optimally aligned sequences, and a portion of the polynucleotide sequence in the comparison region may contain additions or deletions (i.e., gaps) compared to a reference sequence (which does not contain additions or deletions) for the optimal alignment of the two sequences.

Likewise, a polypeptide (protein) with a specific sequence number may include the amino acid sequence of the sequence number or may consist of the amino acid sequence of the sequence number, and so long as the purpose and function of the modified reovirus according to the present invention are maintained, variants of the corresponding amino acid sequence are included within the scope of the present invention. For example, a polypeptide of an amino acid sequence represented by a specific sequence number is a concept that includes functional equivalents (e.g., variants in which some of the amino acid sequence of a polypeptide has been modified by deletion, substitution, or insertion, but are functionally identical to the polypeptide) of the polypeptide molecule thereof. Specifically, a polypeptide indicated by a specific sequence number may include an amino acid sequence having a sequence homology of at least 70%, preferably at least 80%, even more preferably at least 90%, and most preferably at least 95%, with the amino acid sequence indicated by the specific sequence number. For example, a polypeptide having a sequence homology of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% is included. The “% sequence homology” for a polypeptide is determined by comparing a comparison region between two optimally aligned sequences, and a portion of the amino acid sequence in the comparison region may contain additions or deletions (i.e., gaps) compared to a reference sequence (which does not contain additions or deletions) for the optimal alignment of the two sequences.

The modified reovirus according to the present invention may contain the same wild-type base sequence and amino acid sequence as the wild-type reovirus except for the above-mentioned mutations. However, due to the nature of the virus, it is obvious that even in the wild-type sequence, mutations such as deletion, substitution, and/or insertion of some bases or amino acids may occur in the range where the function and characteristics of the virus are maintained. Accordingly, the modified reovirus according to the present invention may include, in addition to the mutation, additional variants of the wild-type sequence which do not impair the function of the virus (effect of inducing production of neutralizing antibodies against antigens, anti-cancer effect, etc.). The genome sequence of the wild-type reovirus according to the present invention is described in detail in the sequence list of this specification.

The modified reovirus according to the present invention may be derived from any wild-type reoviruses and may be a member of the reovirus family which may be obtained from a variety of sources. Preferably, the modified reovirus according to the present invention may be derived from a wild-type human reovirus. Preferably, the wild-type reovirus may be selected from among human reovirus Type 1, human reovirus Type 2, human reovirus Type 3 and human reovirus Type 4. More preferably, the wild-type reovirus may be selected from among human reovirus Type 1 strain Lang, human reovirus Type 2 strain Jones, human reovirus Type 3 strain Dearing and Abney and human reovirus Type 4 strain Ndelle. Most preferably, the wild-type reovirus according to the present invention may be Type 3 reovirus. In addition, The modified reovirus according to the present invention may be derived from one or more reoviruses showing tropism for cells of non-human primates (chimpanzees, gorillas, macaques, monkeys, etc.), rodents (mice, rats, Garyville's rats, hamsters, rabbits, guinea pigs, etc.) and other mammalian species including dogs, cats and common livestock (cattle, horses, pigs, goats), without being limited thereto.

In the present invention, the “exogenous epitope” refers to an exogenous peptide intended to be delivered to cells or the body through reovirus for the purpose of preventing or treating a specific disease. The “epitope (antigenic determinant or antigen group)” refers to a specific part of an antigen identified by antibodies, B cells, T cells, etc., and the immune system can recognize foreign epitopes and trigger an immune response against them. It is obvious that exogenous epitopes are not limited to epitopes of a specific type or sequence, and those skilled in the art can select the desired epitope without limitation depending on the purpose. For example, when trying to achieve the purpose of preventing or treating a specific disease using a reovirus, it is desirable to select an antigenic epitope that is related to the disease. In an embodiment of the present invention, the epitope may be an epitope of an antigen that causes cancer or an infectious disease. Alternatively, diseases (infectious diseases, cancer, etc.) in the present invention may be diseases induced or caused by an antigen containing an epitope according to the present invention. For example, cancer in the present invention may be a cancer that expresses an antigen (tumor antigen) containing an epitope according to the present invention. One or more exogenous epitope-encoding genes may be included in the recombinant vector according to the present invention. When the recombinant vector according to the present invention contains two or more exogenous epitope-encoding genes, the genes may be the same or different types.

In this specification, the “infection” means that a pathogenic microorganism such as a virus invades, develops, and proliferates in the body of a host organism, and settles and proliferates in the tissues, body fluids, and surfaces of humans, animals, and plants, and as a result, the host organism may undergo pathological changes and cause disease.

In the present invention, the “infectious disease” refers to a disease that occurs when pathogenic microorganisms settle and proliferate in the tissues, body fluids, and surfaces of humans, animals, or plants, and can be classified into several types depending on the infection route and whether or not it is contagious. The infection includes viral infections, fungal infections, bacterial infections, protozoal infections and parasitic infections. In the present invention, the infectious diseases may be one or more selected from the group consisting of hepatitis C, influenza, human immunodeficiency virus (HIV)-induced AIDS, tuberculosis, coronavirus disease-19 (COVID-19), and severe acute respiratory syndrome coronavirus 2 (SARS), infantile enteritis caused by rotavirus and non-bacterial acute gastroenteritis caused by norovirus, and according to an embodiment of the present invention, it may be coronavirus infection, without being limited thereto.

For example, the exogenous epitope may be one or more selected from the group consisting of viral antigens, bacterial antigens, fungal antigens, allergens, and tumor antigens.

The virus may be selected from the group consisting of SARS-CoV-2 virus, norovirus, influenza virus, Ebola virus, human papillomavirus, hepatitis B virus, hepatitis C virus, hepatitis D virus, Marburg virus, human parainfluenza virus 1, measles virus, mumps virus, rabies virus, human immunodeficiency virus, dengue fever virus, poliovirus, cytomegalovirus, dengue virus, yellow fever virus, adenovirus, Japanese encephalitis virus, smallpox virus, and Zika virus, and proteins or peptides specific to the virus can be applied as exogenous peptides. For example, the peptide may be selected from among viral envelope proteins, capsid proteins, spike proteins, membrane proteins, receptor binding domains, nucleic acids (DNA or RNA), viral enzymes, hemagglutinin, and the like, but a person skilled in the art can select an appropriate viral protein according to the viral infection for which prevention or treatment is intended.

The present inventors manufactured a recombinant reovirus into which the SARS-CoV-2 epitope (SARS-CoV-2 receptor binding domain) was introduced through specific examples and confirmed its effectiveness in preventing and treating SARS-CoV-2 infection. Accordingly, the recombinant reovirus with the SARS-CoV-2 epitope introduced thereinto may be used for the prevention or treatment of coronavirus disease 19 (COVID-19). For example, the recombinant reovirus with the SARS-CoV-2 epitope introduced thereinto according to the present invention may prevent or treat sepsis, acute respiratory syndrome, pneumonia, cytokine storm, cytokine release syndrome, systemic inflammatory response syndrome, multiple organ failure, pulmonary fibrosis, etc. due to SARS-CoV-2 virus infection. Preferably, the SARS-CoV-2 epitope may be a SARS-CoV-2 receptor binding domain, without being limited thereto. More preferably, the SARS-CoV-2 epitope may be encoded by a polynucleotide containing the nucleic acid sequence of SEQ ID NO: 19 or 22, without being limited thereto.

In the present invention, “coronavirus disease-19 (COVID-19)” is a severe respiratory syndrome caused by SARS-CoV-2. The first case was reported in China in December 2019, and it spread globally, becoming an epidemic. Symptoms of COVID-19 vary but include fever, cough, headache, fatigue, difficulty breathing, and loss of smell and taste. Symptoms appear within 1 to 14 days of being infected with the virus. In particular, one-third of infected people are asymptomatic and do not show any noticeable symptoms. 81% of people who are noticeable enough to be classified as patients develop mild to severe symptoms, 14% of the people develop symptoms such as shortness of breath and hypoxia, and 5% of the people develop serious symptoms such as respiratory failure and shock. Older people are more likely to develop severe symptoms, and organ damage has been observed in some people due to exposure to COVID-19 long after recovery.

SARS-CoV-2 has a nucleotide length of 30 kb and has four important structural proteins; Nucleocapsid (N), Spike(S), Membrane (M), and Envelope (E) proteins. Thereamong, the S protein is an important site for binding to the receptor of a host cell and delivers a viral nucleocapsid into the cell, and replication occurs.

The simplest and most direct way to combat SARS-CoV-2 is to neutralize a virus that enters human cells. That is, SARS-CoV-2 enters cells and replicates, and a new virion is secreted, blocking the mechanism of infecting other cells.

It is known that SARS-CoV-2 is capable of viral replication by binding to the angiotensin-converting enzyme 2 (ACE2) receptor on human cells. Among the S proteins of coronaviruses, the receptor-binding domain (RBD) is a key region that binds to the ACE2 receptor on host cells and contains multiple conformational-dependent epitopes that induce potent neutralizing antibodies against SARS-CoV-2 infection, so it can be a key target in the development of a treatment and vaccine for coronavirus disease-19 (J. Immunol 2005; 174:4908-4915).

In the present invention, a tumor antigen is included without limitation as long as it is specifically expressed in cancer cells or has particularly high expression in cancer cells, is not limited to specific types, and may be selected from among Ovalbumin, CD19, NY-ESO-1, EGFR, TAG72, IL13Rα2 (Interleukin 13 receptor alpha-2 subunit), CD52, CD33, CD20, TSLPR, CD22, CD30, GD3, CD171, NCAM (Neural cell adhesion molecule), FBP (Folate binding protein), Le(Y) (Lewis-Y antigen), PSCA (Prostate stem cell antigen), PSMA (Prostate-specific membrane antigen), CEA (Carcinoembryonic antigen), HER2 (Human epidermal growth factor receptor 2), Mesothelin, CD44v6 (Hyaluronate receptor variant 6), B7-H3, Glypican-3, ROR1 (receptor tyrosine kinase like orphan receptor 1), Survivin, FOLR1 (folate receptor), WT1 (Wilm's tumor antigen), VEGFR2 (Vascular endothelial growth factor 2), tumor virus antigens, TP53, KRAS and the like. A person skilled in the art can select an appropriate tumor antigen known in the art according to the type of cancer to be treated and apply it to the present invention.

The present inventors manufactured a recombinant reovirus, into which ovalbumin antigen (OVA257-264, “OVA antigen”) was introduced, through a specific example, confirmed the immune cell activation function by the virus, and confirmed that the growth of tumors expressing the antigen was prevented and suppressed when the recombinant virus was administered. Accordingly, the recombinant reovirus according to the present invention may be used for the prevention or treatment of cancer through introduction of various tumor antigens.

In addition, the epitope may be one or more selected from the group consisting of CD4⁺ T cells, CD8⁺ T cells, and B-cell epitopes. According to an embodiment of the present invention, the epitope may be an epitope including one or more amino acid sequences selected from the group consisting of SARS-CoV-2 RBD, OVA²⁵⁷⁻²⁶⁴, OVA³²³⁻³³⁹, Adpgk, Rpl18, P15E, S21P2(1), and S21P2(2).

In the present invention, the epitope may form or constitute a fusion protein at the carboxy terminus of the attenuated reovirus Sigma 1 protein, without being limited thereto.

As used herein, the term “recombinant vector” refers to a nucleic acid molecule capable of transporting another linked nucleic acid molecule. Specifically, the vector refers to any medium for the introduction and/or transfer of bases into a host cell in vitro or in vivo, and may be a replication unit (replicon) that can bind other DNA fragments to result in replication of the combined fragment. The “replication unit” refers to any genetic unit (e.g., plasmid, phage, cosmid, chromosome, virus, etc.) that functions as a self-unit of DNA replication in vivo, i.e., is capable of replicating under its own control. The vector includes bacteria, plasmids, phages, cosmids, episomes, viruses, and insertable DNA fragments, i.e., fragments that can be inserted into a host cell genome by homologous recombination, without being limited thereto.

The vector according to the present invention may be composed of double-stranded DNA such as plasmid DNA, linear DNA, hairpin DNA, or minicircle DNA, or may be a recombinant viral vector, but is not limited thereto. The vector may be used without limitation as long as it includes a transposon sequence and a target DNA and can deliver the transposon sequence and the target DNA into a target cell, and a person skilled in the art can select and use a variety of vectors known in the art.

The recombinant vector of the present invention may include preferably a promoter as a transcription initiation factor that RNA polymerase binds to, an optional operator sequence to regulate transcription, an enhancer sequence, a sequence encoding a suitable mRNA ribosome binding site, a sequence regulating the termination of transcription and translation, a terminator, etc., may further include more preferably a polyhistidine tag (amino acid motif consisting of at least 5 histidine residues), a signal peptide gene, an endoplasmic reticulum retention signal peptide, a cloning site, etc. and may further include a marker gene for selection such as a tag gene or an antibiotic resistance gene for selecting transformants, etc. In the recombinant vector, the polynucleotide sequence of each of the genes is operably linked to a promoter. As used herein, the term “operatively linked” refers to a functional linkage between a nucleotide expression control sequence, such as a promoter sequence, and another nucleotide sequence, whereby the regulatory sequence controls the transcription and/or translation of the other nucleotide sequence.

The recombinant vector may be constructed using prokaryotic cells or eukaryotic cells as hosts. For example, when the vector of the present invention is an expression vector and a prokaryotic cell is used as a host, a strong promoter (e.g., pLλ promoter, trp promoter, lac promoter, tac promoter, T7 promoter, etc.) capable of advancing transcription, a ribosome binding site for initiation of translation and a transcription/translation termination sequence are generally included. When using a eukaryotic cell as a host, the origin of replication at which the vector operates in the eukaryotic cell may include the f1 origin of replication, SV40 origin of replication, pMB1 origin of replication, adeno origin of replication, AAV origin of replication and BBV origin of replication, etc., but without being limited thereto. In addition, promoters (e.g., metallothioneine promoter) derived from the genome of mammalian cells or promoters (e.g., adenovirus late promoter, vaccinia virus 7.5K promoter, SV40 promoter, cytomegalovirus promoter and tk promoter of HSV) derived from mammalian viruses may be used, and a polyadenylation sequence is typically included as a transcription termination sequence. In addition, a poly A signal, etc. may be included as a signal sequence, without being limited thereto.

Representative examples of the tag gene may include Avi tag, Calmodulin tag, polyglutamate tag, E tag, FLAG tag, HA tag, His tag (polyhistidine tag), Myc tag, S tag, SBP tag, IgG-Fc tag, CTB tag, Softag 1 tag, Softag 3 tag, Strep tag, TC tag, V5 tag, VSV tag, Xpress tag, and the like. Preferably, the vector according to the invention may include a myc tag. There are no restrictions on the location of the tag in the vector, but it may preferably be located downstream of the mutant S1 gene. Alternatively, the tag may be located on one or both sides of an exogenous epitope-encoding gene, without being limited thereto.

In the present invention, the vector can be delivered into cells through a variety of techniques commonly used to introduce exogenous nucleic acids (DNA or RNA) into prokaryotic or eukaryotic host cells. For example, the vector according to the present invention may be inserted into cells by calcium phosphate coprecipitation; electroporation; microfluidics gene editing; nucleofection; cell squeezing; sonoporation; optical transfection; impalefection; gene gun; magnetofection; viral transduction; DEAE-dextran transfection; lipofection; or transfection with dendrimers, liposomes, or cationic polymers, without being limited thereto.

As used herein, the term “nucleic acid” or “nucleic acid molecule” is meant to comprehensively include DNA (gDNA and cDNA) and RNA molecules, and nucleotides, which are basic structural units in nucleic acids, include not only natural nucleotides but also analogues in which sugar or base sites have been modified. The sequence of a nucleic acid according to the present invention may be modified. The modification includes additions, deletions, or non-conservative or conservative substitutions of nucleotides. The nucleic acid according to the present invention also includes a nucleotide sequence that exhibits substantial identity to the nucleotide sequence. Substantial identity means a nucleotide sequence that exhibits at least 80% homology, more preferably at least 90% homology, and most preferably at least 95% homology, when the nucleotide sequence of the present invention is aligned to correspond as much as possible to any other sequence, and the aligned sequence is analyzed using an algorithm commonly used in the art.

The exogenous epitope-encoding gene is characterized by being located downstream of the mutant S1 gene. Preferably, an exogenous epitope-encoding gene according to the present invention is located downstream of the 251th codon (i.e., stop codon) of the mutant S1 gene and can replace the deleted region of the S1 gene. For example, the exogenous epitope-encoding gene may be substituted or inserted at a position after the 763rd nucleotide (preferably, after the 765th nucleotide) from the 5′ terminal of the base sequence encoding the Sigma 1 protein of the attenuated reovirus, without being limited thereto. That is, the base sequence encoding the epitope may be substituted for the nucleotide sequence at a specific position among 763th (or number 766) to 1416th nucleotides from the 5′ terminal in the base sequence of the S1 gene (e.g., SEQ ID NO: 4 or 5) encoding the Sigma 1 protein of the attenuated reovirus, or may be inserted between nucleotide sequences at a specific position thereamong.

The exogenous epitope-encoding gene according to the present invention may be 10 to 400, 10 to 300, 10 to 250, 10 to 220, 50 to 400, 50 to 300, 50 to 250, 100 to 400, 100 to 300, 100 to 250, or 150 to 250 nucleotide in length, without being limited thereto. In addition, the exogenous epitope-encoding gene may be directly connected to the mutant S1 gene, but may also be connected to the mutant S1 gene via a tag gene or a linker. For example, one or more selected from the group consisting of a linker, Myc protein, FLAG protein, and 2A peptide may be further included before and after the amino acid sequence of the epitope contained at 251st to 455th amino acid positions in the Sigma 1 protein of the attenuated reovirus, but are not limited thereto, and their order is not limited. The 2A peptide may be selected from among 2A peptides including, for example, P2A, T2A, E2A, or F2A, and there is no limitation to the type thereof.

The mutant S1 gene and the exogenous epitope-encoding gene are characterized by being expressed together to produce a fusion protein. That is, the recombinant vector according to the present invention may produce a protein in which the mutated (truncated) Sigma 1 protein and the exogenous epitope are fused through transcription and translation when introduced into a cell. Preferably, the fusion protein may be characterized by replacing the head portion of the wild-type Sigma 1 protein with an exogenous epitope.

Accordingly, the present invention provides a mutant Sigma 1 protein expressed from the recombinant vector and fused with the exogenous epitope. The mutant Sigma 1 protein is characterized by containing the 1st to 250th amino acid sequences of the wild-type reovirus Sigma 1 protein. Preferably, the mutant Sigma 1 protein is characterized by not containing the amino acid sequence after the 250th amino acid of the wild-type reovirus Sigma 1 protein. For example, the mutant Sigma 1 protein may be represented by the amino acid sequence of SEQ ID NO: 1 or 2.

Preferably, the fusion protein generated from the S1 gene and the exogenous epitope-encoding gene is characterized by being expressed as a reovirus capsid. That is, the fusion protein expressed from the recombinant vector according to the present invention may be assembled with other reovirus components and displayed externally as the capsid protein of the recombinant reovirus. After the recombinant reovirus infects a cell, the exogenous epitope located at the head of Sigma 1 protein is exposed to the cell, thereby inducing an immune response to the epitope.

The recombinant vector according to the present invention may further include other reovirus genes required for the production of reovirus in addition to the mutant S1 gene and an exogenous epitope-encoding gene. For example, the recombinant vector may include an essential gene of a reovirus. The essential gene may be selected from the group consisting of L1, L2, L3, M1, M2, M3, S2, S3, and S4, without being limited thereto. Accordingly, the recombinant protein expressed from the mutant S1 gene and the exogenous epitope-encoding gene may be assembled with other reovirus components expressed from reovirus essential genes to form an intact reovirus. Alternatively, the reovirus essential genes may be introduced into a vector separate from the recombinant vector containing the mutant S1 gene and the exogenous epitope-encoding gene and expressed independently of the recombinant vector of the present invention. Here, expression products generated from the respective vectors may be assembled together to form a complete reovirus. Accordingly, the fusion protein expressed from the recombinant vector according to the present invention may be assembled with other reovirus components to form an intact reovirus, so it can be exposed to cells through the viral infection mechanism. The essential genes of reovirus are known, and their sequence information can be confirmed through public databases. For example, sequence information for the L1, L2, L3, M1, M2, M3, S2, S3, and S4 genes of reovirus can be confirmed in GenBank accession numbers EF494435.1, EF494436.1, EF494437.1, EF494438.1, EF494439.1, EF494440, respectively. .1, EF494441.1, EF49442.1, EF494443.1, and EF494444.1, but these are only representative examples, without being limited to this sequence information. In addition, the reovirus gene sequences are specifically provided in the sequence listing herein.

In addition, the present invention provides a cell into which the recombinant vector according to the present invention has been introduced (i.e., transformed, transfected, or transfected). The cell can express a gene inserted into the introduced recombinant vector and produce the mutant Sigma 1 protein with an exogenous epitope fused thereto.

Ultimately, the cell produces a recombinant reovirus containing the mutant Sigma 1 protein fused to the epitope. Accordingly, for the generation of an intact reovirus, the cell may be further introduced with a vector containing one or more genes consisting of the group selected from the group L1, L2, L3, M1, M2, M3, S2, S3, and S4 of the reovirus. Alternatively, the genes may be inserted into and exist in the cell's genome, or may be inserted together in a recombinant vector containing the mutant S1 gene and an exogenous epitope-encoding gene.

In addition, the cell may further contain other components that can assist in the expression of the recombinant protein and the production of the recombinant reovirus. For example, it may further include vaccinia virus capping enzyme, a heterodimer of D1R and D12L, FAST-p10 as a reovirus fusion protein, etc.

In addition, the present invention provides a recombinant reovirus including the mutant S1 gene and an exogenous epitope-encoding gene, wherein the mutant S1 gene has a stop codon at the 251st codon from the start codon. That is, the present invention provides a recombinant reovirus containing the mutant S1 gene according to the present invention and an exogenous epitope-encoding gene in the genome thereof. Accordingly, the recombinant reovirus is characterized by expressing the exogenous epitope. Preferably, the exogenous epitope is located at the head of the mutated (truncated) Sigma 1 protein and is present on the outer capsid of the virus. Preferably, the recombinant reovirus is characterized by being produced from the recombinant vector according to the present invention. That is, the recombinant reovirus may be characterized as containing the fusion protein of mutant sigma 1 and exogenous epitope expressed from the recombinant vector according to the present invention.

The present invention provides a vaccine composition containing the recombinant vector according to the present invention, a cell into which the recombinant vector has been introduced, or reovirus expressed from the recombinant vector as an active ingredient.

In this specification, the “vaccine” is a biological agent containing an antigen that causes an immune response in the living body and refers to an immunogen that creates immunity in a living body by injecting or orally administering it to humans or animals to prevent infections, cancer, etc. The animal is a human or a non-human animal, and the non-human animal refers to a pig, cow, horse, dog, goat, sheep, etc., but is not limited thereto. The immunologically active component of a vaccine may contain appropriate elements of live or dead virus (subunit vaccine), whereby these elements are produced by a purification step of destroying the entire virus or its growth culture and then obtaining a desired structure(s), or, without being limited to, by a synthetic process guided by appropriate manipulation of appropriate systems, such as bacteria, insects, mammals or other species and then isolation and purification processes, or by induction of this synthetic process in animals in need of a vaccine by direct incorporation of genetic material using an appropriate pharmaceutical composition (polynucleotide vaccination).

A vaccine may include one or more of the elements described above and may be prepared by methods known in the art. The vaccine of the present invention may be provided in any form known in the art, for example, in the form of liquid and injection, or in a solid form suitable for suspension, but is not limited thereto, and for example, it may be manufactured in the form of a polypeptide containing the truncated Sigma 1 protein and epitope amino acid sequence of the attenuated reovirus, in the form of a polynucleotide encoding the same, and in the form of a viral vector containing the polynucleotide, without being limited thereto. The vaccine of the present invention may be in any form known in the art, for example, in the form of liquid and injection, or in a solid form suitable for suspension, without being limited thereto. These agents may also be emulsified or encapsulated in a liposome or a fusible glass or prepared in an aerosol or spray form.

The vaccine according to the present invention may, if necessary, contain pharmaceutically acceptable vaccine protective agents, immune enhancers, diluents, absorption accelerators, etc. The vaccine protection agent includes, for example, a lactose phosphate glutamate gelatin mixture, without being limited thereto. When the vaccine is a liquid or injectable formulation, it may contain propylene glycol and sodium chloride in an amount sufficient to prevent hemolysis (e.g., about 1%), if necessary.

There is no particular limitation on a substance used as the immune adjuvant, and the immune adjuvant may be, for example, alum, MPL (monophosphoryl lipid A), aluminum hydroxide, mineral oil or other oil, or an auxiliary molecule added to a vaccine or produced by the body after each induction by the added ingredient, without being limited thereto.

Various exogenous epitopes including the epitope described herein may be introduced into the recombinant reovirus according to the present invention, and upon administration of the reovirus, the immune function of immune cells for the epitope may be improved by inducing stable and repetitive expression of the exogenous epitope, thereby providing a vaccine platform for the prevention or treatment of various diseases. As described above, the disease in one embodiment of the present invention may be a disease associated with the exogenous epitope according to the present invention. For example, the disease may be a disease caused by an antigen containing the epitope, and when the disease is cancer, the cancer may be a cancer that expresses a tumor antigen containing the exogenous epitope according to the present invention.

For example, the vaccine composition according to the present invention may achieve the following characteristics through the recombinant reovirus:
(a) induce the expression of the exogenous epitope in cells infected with the recombinant reovirus;
(b) induce the presentation of the exogenous epitope by antigen-presenting cells;
(c) activate cytokine production in T cells by the exogenous epitope; and
(d) induce the production of a neutralizing antibody against the exogenous epitope.

In addition, One or more epitopes introduced into the recombinant reovirus of the present invention may cause an immune response by allowing T cells or B cells to identify antigens, producing neutralizing antibodies or inducing cellular immunity in a host, without being limited thereto.

In the present invention, the “neutralizing antibody” means any antibody or antigen-binding fragment thereof that binds to a pathogen and interferes with the pathogen's ability to infect cells or cause disease.

In the present invention, the “cellular immunity” refers to an immune activity in which a lymphoid cell directly mounts an immune response to an antigen. It refers to an immune activity in which white blood cells act on an antigen to phagocytose the cells or cause a toxic cell reaction to remove the antigen. In the body, it constitutes the immune system along with humoral immunity carried out by serum antibodies, and a representative example is the action of cytotoxic T cells. T cells combine with B cells to form an antibody and then directly contact the antigen to destroy the antigen.

In addition, the present invention provides a pharmaceutical composition for preventing or treating diseases, including the recombinant vector according to the present invention, a cell into which the vector has been introduced, or a reovirus to be expressed from the recombinant vector as an active ingredient. Here, the mutant S1 gene is characterized by having a stop codon at the 251st codon from the start codon, and the diseases are characterized by being associated with the epitope.

Specifically, the recombinant reovirus according to the present invention may be used to prevent or treat infectious diseases caused by the virus by introducing various viral epitopes. That is, the present invention provides a pharmaceutical composition for preventing or treating viral infections including a recombinant vector including a mutant S1 gene of reovirus, and a viral epitope-encoding gene; a cell into which the recombinant vector has been introduced; or recombinant reovirus produced from the cell as an active ingredient, wherein the mutant S1 gene has a stop codon at the 251st codon from the start codon thereof.

In addition, the recombinant reovirus according to the present invention may be used to prevent or treat cancer by introducing various tumor antigens. That is, the present invention provies a pharmaceutical composition for preventing or treating cancer, including a recombinant vector including a mutant S1 gene of reovirus and an epitope-encoding gene of a tumor antigen; a cell into which the recombinant vector has been introduced; or recombinant reovirus produced from the cell as an active ingredient, wherein the mutant S1 gene has a stop codon at the 251st codon from the start codon thereof.

The cells may be mammalian cells, but are not limited thereto.

The viral epitope and tumor antigen according to the present invention have been described above.

The term “cancer” used herein is characterized by uncontrolled cell growth. Due to this abnormal cell growth, a cell mass called a tumor is formed, which infiltrates surrounding tissues and, in severe cases, metastasizes to other organs of the body. In the present invention, the cancer may be solid cancer or a blood cancer and may be selected from among squamous cell carcinoma, glioma, lung cancer, adenocarcinoma of the lung, peritoneal cancer, skin cancer, eye cancer, rectal cancer, perianal cancer, esophageal cancer, small intestine cancer, endocrine cancer, parathyroid cancer, adrenal cancer, osteosarcoma, soft tissue sarcoma, urethral cancer, blood cancer, liver cancer, gastrointestinal cancer, pancreatic cancer, glioblastoma, cervical cancer, ovarian cancer, bladder cancer, breast cancer, colon cancer, colorectal cancer, endometrial cancer, uterine cancer, salivary gland cancer, kidney cancer, prostate cancer, vulva cancer, thyroid cancer, head and neck cancer, oral cancer, tongue cancer, brain cancer and stromal tumor. The blood cancer may be leukemia, lymphoma, multiple myeloma, etc. Preferably, the skin cancer may be selected from squamous cell carcinoma, basal cell carcinoma, and melanoma. Preferably, the melanoma may be metastatic melanoma. In an embodiment of the present invention, the cancer may be a cancer that expresses or does not express PD-L1. In another embodiment of the present invention, the cancer may be a cancer having a mutation in a cancer development suppressor gene (p53, Rb, etc.) or a RAS activation mutation. More preferably, the cancer according to the present invention may be a cancer resistant to wild-type reovirus.

The content of the recombinant vector, the cell, or the recombinant reovirus in the composition of the present invention may be appropriately adjusted depending on the symptoms of diseases, the degree of progression of the symptoms, the patient's condition, etc., and, for example, may be 0.0001 to 99.9% by weight, or 0.001 to 50% by weight based on the total weight of the composition, without being limited thereto. The content ratios are values based on a dry amount from which a solvent has been removed.

The pharmaceutical composition according to the present invention may further include an appropriate carrier, excipient, and diluent commonly used in the preparation of pharmaceutical compositions. The excipient may be, for example, one or more selected from the group consisting of a diluent, a binder, a disintegrant, a lubricant, an adsorbent, a humectant, a film-coating material, and a controlled-release additive.

The pharmaceutical composition according to the present invention may be formulated and used in the form of external preparations such as powders, granules, sustained-release granules, enteric-coated granules, liquid formulations, eye drops, Elsilic, emulsions, suspensions, spirits, troches, fragrances, limonadese, tablets, sustained-release tablets, enteric-coated tablets, sublingual tablets, hard capsules, soft capsules, sustained-release capsules, enteric capsules, pills, tinctures, soft extracts, dry extracts, liquid extracts, injections, capsules, irrigation fluids, warning agents, lotions, pasta products, sprays, inhalants, patches, sterile injection solutions or aerosols according to a method of each thereof, and the external preparations may have formulations such as creams, gels, patches, sprays, ointments, warning agents, lotions, liniment agents, pasta agents, or cataplasma agents.

Examples of a carrier, excipient and diluent which may be included in the pharmaceutical composition according to the present invention include lactose, dextrose, sucrose, oligosaccharides, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, mineral oil, and the like.

When formulated, it is prepared using a diluent or excipient such as a commonly used filler, extenders, binder, wetting agent, disintegrant, and surfactant.

Examples of an additive to a tablet, powder, granule, capsule, pill, and troch according to the present invention includes an excipient such as corn starch, potato starch, wheat starch, lactose, white sugar, glucose, fructose, di-mannitol, precipitated calcium carbonate, synthetic aluminum silicate, calcium monohydrogen phosphate, calcium sulfate, sodium chloride, sodium bicarbonate, purified lanolin, microcrystalline cellulose, dextrin, sodium alginate, methylcellulose, sodium carboxymethylcellulose, kaolin, urea, colloidal silica gel, hydroxypropyl starch, hydroxypropylmethylcellulose (HPMC) 1928, HPMC 2208, HPMC 2906, HPMC 2910, propylene glycol, casein, calcium lactate or Primogel; a binder such as gelatin, gum arabic, ethanol, agar powder, cellulose acetate phthalate, carboxymethyl cellulose, calcium carboxymethyl cellulose, glucose, purified water, sodium caseinate, glycerin, stearic acid, sodium carboxymethyl cellulose, sodium methyl cellulose, methyl cellulose, microcrystalline cellulose, dextrin., hydroxycellulose, hydroxypropyl starch, hydroxymethylcellulose, refined shellac, starch, hydroxypropylcellulose, hydroxypropylmethylcellulose, polyvinyl alcohol or polyvinylpyrrolidone; a disintegrant such as hydroxypropyl methyl cellulose, corn starch, agar powder, methyl cellulose, bentonite, hydroxypropyl starch, sodium carboxymethyl cellulose, sodium alginate, calcium carboxymethyl cellulose, calcium citrate, sodium lauryl sulfate, silicic acid anhydride, 1-hydroxy propylcellulose, dextran, ion exchange resin, polyvinyl acetate, formaldehyde-treated casein and gelatin, alginic acid, amylose, guar gum, sodium bicarbonate, polyvinylpyrrolidone, calcium phosphate, gelled starch, gum arabic, amylopectin, pectin, sodium polyphosphate, ethyl cellulose, white sugar, magnesium aluminum silicate, di-sorbitol solution or light anhydrous silicic acid; and a lubricant such as calcium stearate, magnesium stearate, stearic acid, hydrogenated vegetable oil, talc, lycopodium, kaolin, petrolatum, sodium stearate, cacao fat, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohol, silicone oil, paraffin oil, polyethylene glycol fatty acid ether, starch, sodium chloride, sodium acetate, sodium oleate, dl-leucine or light anhydrous silicic acid.

As an additive of the liquid formulation according to the present invention, water, dilute hydrochloric acid, dilute sulfuric acid, sodium citrate, sucrose monostearate, polyoxyethylene sorbitol fatty acid esters (twin esters), polyoxyethylene monoalkyl ethers, lanolin ethers, lanolin esters, acetic acid, hydrochloric acid, aqueous ammonia, ammonium carbonate, potassium hydroxide, sodium hydroxide, prolamine, polyvinylpyrrolidone, ethyl cellulose, sodium carboxymethyl cellulose, or the like may be used.

As a syrup according to the present invention, a solution of white sugar, other sugars or sweeteners, or the like may be used, and a flavoring agent, a colorant, a preservative, a stabilizer, a suspending agent, an emulsifier, a viscous agent, or the like may be used as needed.

Purified water may be used as an emulsion according to the present invention, and an emulsifier, a preservative, a stabilizer, a flavoring agent, etc. may be used as needed.

As a suspending agent according to the present invention, a suspending agent such as acacia, tragacanth, methyl cellulose, carboxymethyl cellulose, sodium carboxymethyl cellulose, microcrystalline cellulose, sodium alginate, hydroxypropylmethylcellulose (HPMC), HPMC 1828, HPMC 2906, HPMC 2910, or the like may be used, and a surfactant, a preservative, a stabilizer, a colorant, and a flavoring agent may be used as needed.

Examples of an injection according to the present invention may include a solvent such as distilled water for injection, 0.9% sodium chloride injection, IV injection, dextrose injection, dextrose+sodium chloride injection, PEG, lactated IV injection, ethanol, propylene glycol, non-volatile oil-sesame oil, cottonseed oil, peanut oil, soybean oil, corn oil, ethyl oleate, isopropyl myristate, or benzene benzoate; a solubilizing agent such as sodium benzoate, sodium salicylate, sodium acetate, urea, urethane, monoethylacetamide, butazolidine, propylene glycol, twins, nijuntinamide, hexamine, or dimethylacetamide; a buffer such as weak acids and their salts (acetic acid and sodium acetate), weak bases and their salts (ammonia and ammonium acetate), organic compounds, proteins, albumin, peptone, or gums; a tonicity agent such as sodium chloride; a stabilizer such as sodium bisulfite (NaHSO₃) carbon dioxide gas, sodium metabisulfite (Na₂S₂O₅), sodium sulfite (Na₂SO₃), nitrogen gas (N₂), or ethylenediamine tetraacetic acid; a sulfating agent such as sodium bisulfide 0.1%, sodium formaldehyde sulfoxylate, thiourea, ethylenediamine disodium tetraacetate, and acetone sodium bisulfite; an analgesic such as benzyl alcohol, chlorobutanol, procaine hydrochloride, glucose, and calcium gluconate; a suspending agent such as SiMCsodium, sodium alginate, Tween 80, and aluminum monostearate and the like.

As a suppository according to the present invention, a base such as cacao butter, lanolin, witapsol, polyethylene glycol, glycerogelatin, methyl cellulose, carboxymethyl cellulose, mixture of stearic acid and oleic acid, subanal, cottonseed oil, peanut oil, palm oil, cacao butter+cholesterol, lecithin, lanet wax, glycerol monostearate, tween or span, imhausen, monolen(propylene glycol monostearate), glycerin, adeps solidus, Buytyrum Tego-G, cebes pharma 16, hexalide base 95, cotomar, hydrokote SP, S-70-XXA, S-70-XX75 (S-70-XX95), hydrokote 25, hydrokote 711, idropostal, massa estrarium (A, AS, B, C, D, E, I, T), martha-MF, marsupol, marsupol-15, neosupostal-N, paramound-B, suposiro (OSI, OSIX, A, B, C, D, H, L), suppository type IV (AB, B, A, BC, BBG, E, BGF, C, D, 299), supostal (N, Es), wecobi (W, R, S, M, Fs), or testester triglyceride base (TG-95, MA, 57) may be used.

Examples of solid formulations for oral administration may include tablets, pills, powders, granules, capsules, etc., and these solid formulations may be prepared by mixing the extract with at least one excipient, for example, starch, calcium carbonate, sucrose or lactose, gelatin, or the like. In addition to simple excipients, lubricants such as magnesium stearate and talc may also be used.

Examples of a liquid formulation for oral administration may include suspensions, internal solutions, emulsions, syrups, etc., and various excipients such as wetting agents, sweeteners, flavoring agents, and preservatives may be included in addition to water and liquid paraffin which are commonly used simple diluents. Examples of a formulation for parenteral administration may include sterilized aqueous solutions, non-aqueous solvents, suspensions, emulsions, freeze-dried formulations, and suppositories. Propylene glycol, polyethylene glycol, vegetable oils such as olive oil, injectable esters such as ethyl oleate, or the like may be used as a non-aqueous solvent and a suspending agent.

The pharmaceutical composition according to the present invention may be administered in a pharmaceutically effective amount. In the present invention, the “pharmaceutically effective amount” means an amount sufficient to treat a disease with a reasonable benefit/risk ratio applicable to medical treatment, and the effective amount may be determined according to the type of disease, the severity of disease, the activity of drug, the sensitivity to drug, the time of administration, the route of administration and the excretion rate, the duration of treatment, factors including drugs used concurrently, and other factors well known in the medical field.

The pharmaceutical composition according to the present invention may be administered as an individual therapeutic agent or in combination with other therapeutic agents, and may be administered sequentially or concurrently with an existing therapeutic agent and once or multiple times. It is important to administer the pharmaceutical composition in an amount to obtain the maximum effect with the minimum amount without side effects by considering all the above-mentioned factors, and such an amount may be easily determined by a person having ordinary skill in the technical field to which the present invention pertains.

The pharmaceutical composition of the present invention may be administered to a subject by various routes. All methods of administration may be expectable and may include, for example, oral administration, subcutaneous injection, intraperitoneal administration, intravenous injection, intramuscular injection, injection in the paraspinal space (intrathecal), sublingual administration, buccal administration, insertion into the rectum, insertion into the vagina, intraocular administration, ear administration, nasal administration, inhalation, spraying through the mouth or nose, dermal administration, transdermal administration, and the like.

The pharmaceutical composition of the present invention may be determined according to the type of drug as an active ingredient together with various related factors such as the type of disease to be treated, the route of administration, the patient's age, the patient's gender, the patient's weight, and the severity of disease. Specifically, the effective amount of the composition according to the present invention may vary depending on the patient's age, gender, and weight. Generally, 0.001 to 150 mg, preferably 0.01 to 100 mg, per 1 kg of body weight may be administered daily, every other day, or in divided doses from once to three times a day. However, since the amount may increase or decrease depending on the route of administration, the severity of disease, gender, weight, age, etc., it does not limit the scope of the present invention in any way.

In the present invention, the “subject” means a subject in need of treatment of a disease, and, more specifically, a mammal such as a human or non-human primate, mouse, rat, dog, cat, horse, and cow.

In the present invention, the “administering” means providing the predetermined composition according to the present invention to a subject by any suitable method.

In the present invention, the “prevention” refers to all actions to suppress or delay the onset of a target disease, the “treatment” refers to all actions to improve or beneficially change the target disease and the resulting metabolic abnormality through the pharmaceutical composition according to the present invention, and the “alleviation” refers to all actions to reduce a parameter related to the target disease, e.g., the severity of a symptom, by the composition according to the present invention.

The above description of the pharmaceutical composition is also applied to a vaccine composition.

In addition, the present invention provides a food composition for preventing or alleviating various infectious diseases, allergies or cancers, including the recombinant vector containing a mutant S1 gene of reovirus and an exogenous epitope-encoding gene; a cell into which the recombinant vector has been introduced; or recombinant reovirus produced from the cell as an active ingredient, wherein the mutant S1 gene has a stop codon at the 251st codon from the start codon thereof.

When the recombinant vector of the present invention, the cell into which the vector has been introduced, or the recombinant reovirus is used as a food additive, the vector, the cell, or the recombinant reovirus may be added as is or used together with other foods or food ingredients, and may be used appropriately according to conventional methods. The mixing amount of active ingredients may be appropriately determined according to the purpose of use (e.g., prevention, health care, or therapeutic treatment). In general, when preparing food or beverages, the vector, cell, or recombinant reovirus of the present invention may be added in an amount of 15% by weight or less or 10% by weight or less based on the raw material. However, the amount may be within or below the ranges when consumed for a long period for the purpose of health care and hygiene or health condition control, and active ingredients may be used in an amount equal to or larger than the above-mentioned amount since there is no problem in terms of safety.

There is no particular limitation in the type of the foods. Examples of foods to which the substances can be added may include meat, sausage, bread, chocolate, candy, snacks, confectionery, pizza, ramen, other noodles, gum, dairy products including ice cream, various soups, beverages, tea, drinks, alcoholic beverages, vitamin complexes, etc., and may include all of healthy functional foods in the usual sense.

The health beverage composition according to the present invention may contain various flavoring agents, natural carbohydrates, etc. as additional components, as in conventional beverages. The aforementioned natural carbohydrates may be monosaccharides such as glucose and fructose, disaccharides such as maltose and sucrose, polysaccharides such as dextrins and cyclodextrins, and sugar alcohols such as xylitol, sorbitol, and erythritol. As sweeteners, natural sweeteners such as thaumatin and stevia extracts, synthetic sweeteners such as saccharin and aspartame, etc. may be used. The proportion of the natural carbohydrates per 100 mL of the composition of the present invention may be generally about 0.01 to 0.20 g or 0.04 to 0.10 g.

In addition to the above-mentioned substances, the composition of the present invention may contain various nutrients, vitamins, electrolytes, flavors, colorants, pectic acid and its salts, alginic acid and its salts, organic acids, protective colloidal thickeners, pH adjusters, stabilizers, preservatives, glycerin, alcohol, carbonating agents used for carbonated beverages, and the like. Furthermore, the composition of the present invention may contain fruit flesh for preparing natural fruit juice, fruit juice beverages, and vegetable beverages. These components may be used individually or in combination. The proportion of these additives may not be critical, but may generally be within the range of 0.01 to 0.20 parts by weight per 100 parts by weight of the composition of the present invention.

In this specification, the “health functional food” is the same term as food for special health use (FoSHU), and refers to food with high medical and medical effects that has been processed to efficiently exhibit bioregulatory functions in addition to supplying nutrients. This food may be produced in various forms such as tablets, capsules, powders, granules, liquids, pills, etc. to achieve useful effects in preventing or improving infectious diseases, allergies, and/or cancers.

The healthy functional food of the present invention may be produced by methods commonly used in the industry field, and may be produced by adding raw materials and ingredients commonly added in the industry field during production. In addition, unlike general drugs, it is made from food, so it has the advantage of not having any side effects that may occur when taking drugs for a long time, and it can be highly portable.

In addition, the present invention provides a method of manufacturing the reovirus-based vaccine composition according to the present invention, the method including the following steps:
(S1) a step of manufacturing a recombinant vector including a mutant S1 gene of reovirus; and
(S2) a step of inserting an exogenous epitope-encoding gene downstream of the mutant S1 gene or replacing a downstream sequence of the mutant S1 gene with an exogenous epitope-encoding gene.

The recombinant vector may be a reverse-evolution viral vector, without being limited thereto. The reverse-evolution viral vector may refer to a viral vector manufactured to produce S1 segment RNA encoding the attenuated reovirus sigma 1 protein in a host cell, and may be produced through methods known in the art. The vector is constructed such that the viral RNA segment can be transcribed from the T7 RNA polymerase promoter, and the 3′ end is naturally formed by the ribozyme in the vector, so the viral RNA is produced in T7 polymerase-expressing cells into which the vector is introduced, and viral proteins are synthesized using this. This is called a reverse genetics system. This is a useful method for designing and creating mutant strains of RNA viruses.

The manufacturing method may include (S3) a step of introducing the recombinant vector manufactured in the step (S2) into a cell after the step (S2), without being limited thereto. The cell may be preferably a cell. Alternatively, the cell may be one or more selected from the group consisting of BHK21, L929, HEK293, CHO, PER.C6, HeLa, and Vero cell, without being limited thereto. The virus introduced into the cell can replicate and proliferate by expressing a viral gene within the cell.

Throughout the specification of the present invention, when a part is said to “comprise” a certain component, this means that it may further include other components rather than excluding other components unless specifically stated to the contrary. The terms “about,” “substantially,” and the like used in this specification are used to mean at or close to a presented numerical value when manufacturing and material tolerances inherent in stated meaning are presented, and are used to prevent unscrupulous infringers from unfairly exploiting the invention in which precise or absolute figures are mentioned to aid understanding of the embodiments.

Throughout this specification, the term “a combination thereof” included in the Markushi format expression refers to a mixture or combination of one or more elements selected from the group consisting of components described in the Markushi format expression, and to include one or more selected from the group consisting of the components.

Now, the present invention will be described in more detail with reference to the following preferred examples. These examples are provided for illustrative purposes only and should not be construed as limiting the scope and spirit of the present invention.

### EXAMPLE

### Example 1. Manufacturing Principle of Reovirus-Based Vaccine Platform

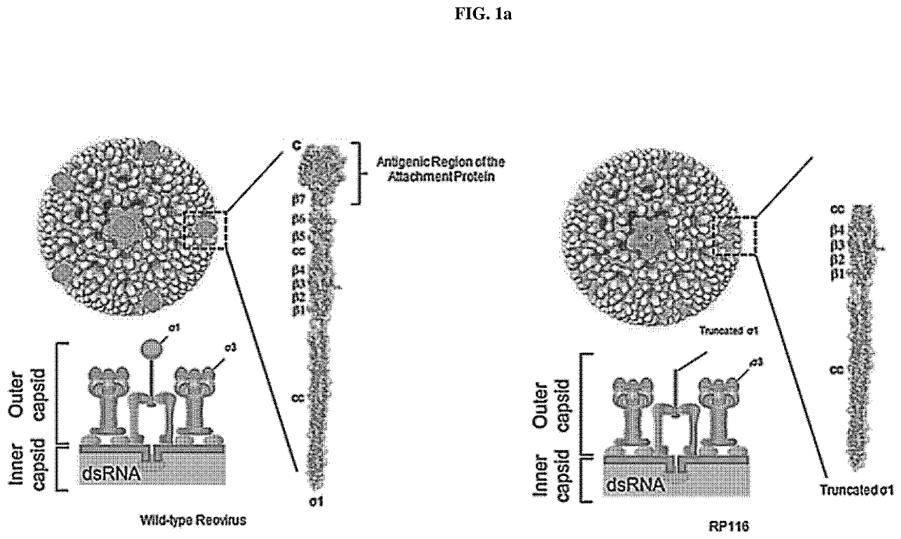

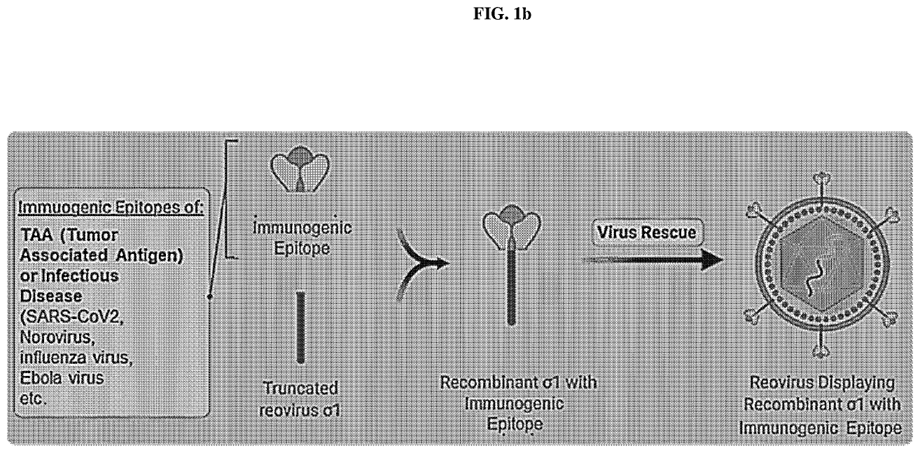

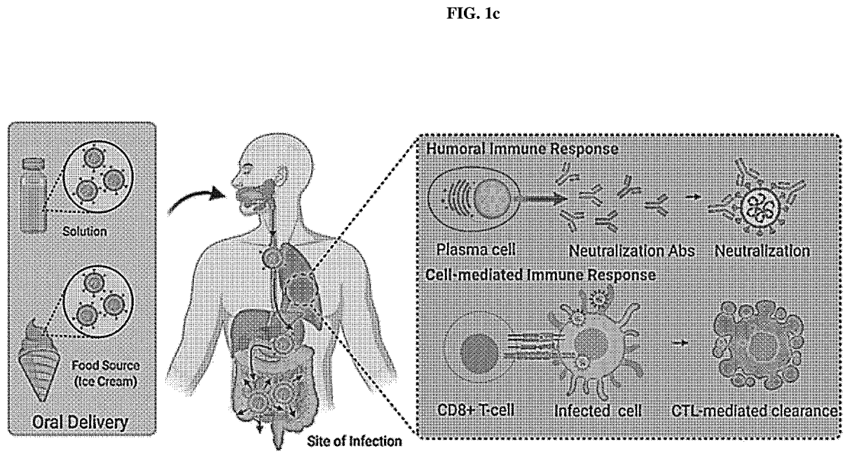

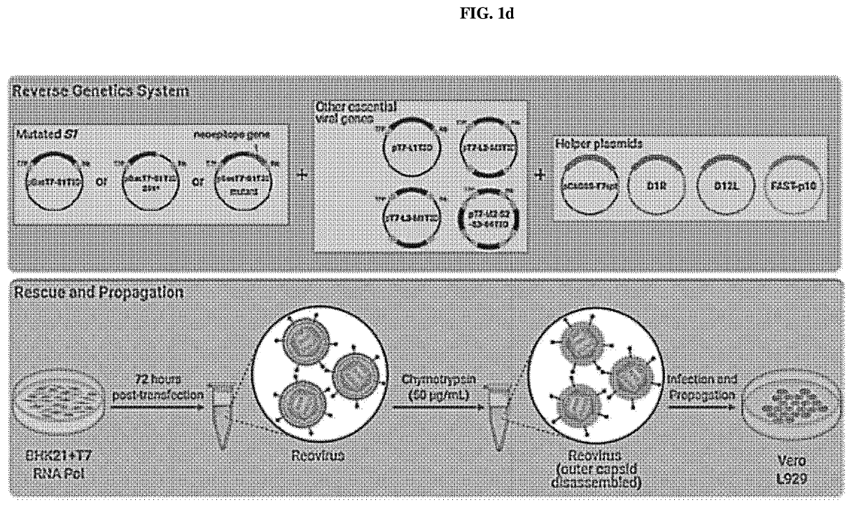

Sigma 1 protein (σ1 protein) is a wild-type (WT) reovirus outer capsid protein. Reovirus enters cells by recognizing and binding to JAM-A, a protein on the surface of target cells, through the σ1 protein. Reovirus RP116 is a reovirus mutation in which the 763rd nucleotide of the S1 segment (S1 gene), which expresses the σ1 protein, is substituted from C to T. Due to a substitution of the nucleotide, RP116 has a unique mutation in which CAA (amino acid 251Q) is replaced by 763TAA (STOP codon). Accordingly, RP116 produces a shorter σ1 protein compared to the σ1 protein produced by the wild-type reovirus. RP116 can be produced at a high titer and has high stability, so it can be used for oncolytic immunotherapy through intratumoral, intravenous, and/or oral administration (FIG. 1a).

Production of the unique σ1 protein of RP116 suggests that the globular head of the σ1 protein of the wild-type reovirus is not essential for viral replication. Accordingly, the present inventors predicted that the globular head of the σ1 protein could be replaced with antigenic fragments from a variety of other pathogens. So, the present inventors attempted to manufacture an innovative and safe reovirus vaccine platform that can be manufactured in a variety of ways by adding an antigenic site of 200 amino acids or more to RP116-derived σ1 protein (FIG. 1b). In particular, the non-enveloping reovirus is stable even under harsh environmental conditions and, accordingly, has the advantage of being able to be administered in the form of beverages or food. When administered orally, reovirus preferentially infects M cells in the small intestine, and various tumor-specific antigens and pathogens recombined with ø1 protein can induce an immune response to the antigen (FIG. 1c).

The present inventors manufactured a vaccine platform using the quick exchange technology based on the reverse genetics system of reovirus. The reverse genetics system of reovirus consists of vectors carrying individual fragments of a total of 10 viral genes (Kobayshi et al., 2007) or multiple gene fragments (Kobayashi et al., 2010). The present inventors used the pUC19 vector as a vector for inserting the reovirus-derived gene in a specific example, but this is only a representative example and the present invention is not limited thereto. For example, pS1Att, pS1XX, pL1, pSet2, pSet3, pSet4, and the like may be used as vectors to express the Sigma 1 protein of the reovirus according to the present invention. In cells into which the recombinant vector is introduced together with T7 RNA polymerase, a viral gene is transcribed, ultimately producing reovirus particles capable of replication within 24 to 72 hours. The region from 251st to the C-terminus (i.e., 251st to 455th amino acids of the σ1 protein of the wild-type reovirus) can be conveniently replaced with an antigen of a non-reovirus by designing and introducing a restriction enzyme recognition site after (i.e. downstream) the STOP codon mutation (251st amino acid position of the σ1 protein of the wild-type reovirus) in the S1 segment of RP116. For example, Myc tag and FLAG tag for detection purposes can be combined after the 250th codon of the mutated S1 gene, and various foreign epitopes can be fused. In addition, co-transfection of an auxiliary vector, such as a vaccinia virus capping enzyme, a heterodimer of D1R and D12L, or FAST-p10 as a reovirus fusion protein, can further improve the production rate of recombinant reovirus. In addition, when chymotrypsin treatment is applied before virus infection, attachment and entry of the virus to production cell lines (BHK, L929, Vero cell, etc.) can be made easier (FIG. 1d).

The sigma 1 protein (representing two sequences) of the modified reovirus (RP116) used in the examples of the present invention and the gene sequence encoding the protein are shown in Table 1 below. In addition, the amino acid sequence of the epitope, the construct amino acid sequence, and the codon-optimized construct DNA sequence, which were used in the examples of the present invention, are shown in Table 2 below.

TABLE 1

| | | SEQ |
|---|---|---|
| Classifi- | | ID |
| cation | Sequence | NO: |
| RP116 | MDPRLREEVVRLIIALTSDNGVSLSKGLES | 1 |
| Sigma-1 | RVSALEKTSQIHSDTILRITQGLDDANKRI | |
| protein | IALEQSRDDLVASVSDAQLAISRLESSIGA | |
| (1) | LQTVVNGLDSSVTQLGARVGQLETGLAELR | |
| | VDHDNLVARVDTAERNIGSLTTELSTLTLR | |
| | VTSIQADFESRISTLERTAVTSAGAPLSIR | |
| | NNRMTMGLNDGLXLSGNNLAIRLPGNTGLN | |
| | IQNGGLQFRFNTDQFQIVNNNLTLKTTVFD | |
| | SINSRXGAXE | |
| RP116 | MDPRLREEVVRLIIALTSDNGASLSKGLES | 2 |
| Sigma-1 | RVSALEKTSQIHSDTILRITQGLDDANKRI | |
| protein | IALEQSRDDLVASVSDAQLAISRLESSIGA | |
| (2) | LQTVVNGLDSSVTQLGARVGQLETGLAELR | |
| | VDHDNLVARVDTAERNIGSLTTELSTLTLR | |
| | VTSIQADFESRISTLERTAVTSAGAPLSIR | |
| | NNRMTMGLNDGLTLSGNNLAIRLPGNTGLN | |
| | IQNGGLQFRFNTDQFQVVNNNLTLKTTVFD | |
| | SINSRIGATE | |
| RP116 | GCTATTGGTCGGATGGATCCTCGCCTACGT | 5 |
| Sigma-1 | GAAGAAGTAGTACGGCTGATAATCGCATTA | |
| gene (full | ACGAGTGATAATGGAGTATCACTGTCAAAA | |
| length) | GGGCTTGAATCAAGGGTCTCGGCGCTCGAG | |
| | AAGACGTCTCAAATACACTCTGATACTATC | |
| | CTCCGGATCACCCAGGGACTCGATGATGCA | |
| | AACAAACGAATCATCGCTCTTGAGCAAAGT | |
| | CGGGATGACTTGGTTGCATCAGTCAGTGAT | |
| | GCTCAACTTGCAATCTCCAGATTGGAAAGC | |
| | TCTATCGGAGCCCTCCAAACAGTTGTCAAT | |
| | GGACTTGATTCGAGTGTTACCCAGTTGGGT | |
| | GCTCGAGTGGGACAACTTGAGACAGGACTT | |
| | GCAGAGCTACGCGTTGATCACGACAATCTC | |
| | GTTGCGAGAGTGGATACTGCAGAACGTAAC | |
| | ATTGGATCATTGACCACTGAGCTATCAACT | |
| | CTGACGTTACGAGTAACATCCATACAAGCG | |
| | GATTTCGAATCTAGGATATCCACATTAGAG | |
| | CGCACGGCGGTCACTAGCGCGGGAGCTCCC | |
| | CTCTCAATCCGTAATAACCGTATGACCATG | |
| | GGATTAAATGATGGACTCAYGTTGTCAGGG | |
| | AATAATCTCGCCATCCGATTGCCAGGAAAT | |
| | ACGGGTCTGAATATTCAAAATGGTGGACTT | |
| | CAGTTTCGATTTAATACTGATCAATTCCAG | |
| | ATAGTTAATAATAACTTGACTCTCAAGACG | |
| | ACTGTGTTTGATTCTATCAACTCAAGGABA | |
| | GGCGCAAYTGAGTAAAGTKMCGTGGCGTCG | |
| | GCAGTGACTCCCTTGAGATTAAACAGTAGC | |
| | ACGAAGGTGCTGGATATGCTAATAGACAGT | |
| | TCAACACTTGAAATTAATTCTAGTGGACAG | |
| | CTAACTGTTAGATCGACATCCCCGAATTTG | |
| | AGGTATCCGATAGCTGATGTTAGCGGCGGT | |
| | ATCGGAATGAGTCCAAATTATAGGTTTAGG | |
| | CAGAGCATGTGGATAGGAATTGTCTCCTAT | |
| | TCTGGTAGTGGGCTGAATTGGAGGGTACAG | |
| | GTGAACTCCGACATTTTTATTGTAGATGAT | |
| | TACATACATATATGTCTTCCAGCTTTTGAC | |
| | GGTTTCTCTATAGCTGACGGTGGAGATCTA | |
| | TCGTTGAACTTTGTTACCGGATTGTTACCA | |
| | CCGTTACTTACAGGAGACACTGAGCCCGCT | |
| | TTTCATAATGACGTGGTCACATATGGAGCA | |
| | CAGACTGTAGCTATAGGGTTGTCGTCGGGT | |
| | GGTGCGCCTCAGTATATGAGTAAGAATCTG | |
| | TGGGTGGAGCAGTGGCAGGATGGAGTACTT | |
| | CGGTTACGTGTTGAGGGGGGTGGCTCAATT | |
| | ACGCACTCAAACAGTAAGTGGCCTGCCATG | |
| | ACCGTTTCGTACCCGCGTAGTTTCACGTGA | |
| | GGATCAGACCACCCCGCGGCACTGGGGCAT | |
| | TTCATC | |

TABLE 2

| | | Construct | Codon Optimized |
|---|---|---|---|
| Epitope | Amino Acid | Amino Acid | Construct DNA |
| Name | Sequence | Sequence | Sequence (5'-3') |
| RBD | NITNLCPFGEVFNAT | DLCFTNVYADSFNYN | aatatcactaacttg |
| | RFASVYAWNRKRISN | YLYRLFRKSNITNLC | tgtccgttcggcgag |
| | CVADYSVLYNSASFS | PFGEVFNATRFASVY | gtttttaatgcgacc |
| | TFKCYGVSPTKLNDL | AWNRKRISNCVADYS | aggtttgcttccgtg |
| | CFTNVYADSFVIRGD | VLYNSASFSTFKCYG | tacgcctggaacagg |
| | EVRQIAPGQTGKIAD | VSPTKLNVIRGDEVR | aaacggatctccaat |
| | YNYKLPDDFTGCVIA | QIAPGQTGKIADYNY | tgtgtcgccgattac |
| | WNSNNLDSKVGGNYN | KLPDDFTGCVIAWNS | tccgtcttgtataat |
| | YLYRLFRKSNLKPFE | NNLDSKVGGNLKPFE | tcagcatctttcagc |
| | RDISTEIYQAGSTPC | RDISTEIYQAGSTPC | acgtttaaatgttac |
| | NGVEGFNCYFPLQSY | NGVEGFNCYFPLQSY | ggagtttcccccaca |
| | GFQPTNGVGYQPYRV | GFQPTNGVGYQPYRV | aaattgaatgacctt |
| | VVLSFELLHAPATV | VVLSFELLHAPATV | tgctttacgaacgtc |
| | | | tacgcggattcattt |
| | | | gtaatccggggggac |
| | | | gaagttaggcaaatt |
| | | | gcgccagggcagact |
| | | | ggcaagatagctgac |
| | | | tataattataaattg |
| | | | ccggatgactttacg |
| | | | ggctgtgtgattgct |
| | | | tggaactcaaataat |
| | | | ctggactcaaaggta |
| | | | gggggaaattataac |
| | | | tacctttacaggctg |
| | | | ttccggaagagtaat |
| | | | ctgaagccattcgaa |
| | | | agagatataagtaca |
| | | | gagatctaccaagct |
| | | | ggaagcaccccctgc |
| | | | aatggtgttgaagga |
| | | | ttcaattgttatttc |
| | | | ccattgcaatcctat |
| | | | ggttttcaaccgacg |
| | | | aatggggtgggatac |
| | | | caaccatatcgagtt |
| | | | gtggttctcagtttc |
| | | | gagttgcttcatgct |
| | | | cctgcgacagtatgt |
| | | | ggaccaaaaaaatct |
| | | | actaatctggtgaag |
| | | | aataaatgcgtcaat |
| | | | ttttaa |
| OVA²⁵⁷⁻²⁶⁴ | SIINFEKL | GGGGSGGGGSEQKLI | aagctggattataag |
| | | SEEDLSIINFEKLGG | gatgacgacgataag |
| | | GGSSIINFEKLGGGG | tgaggaggtggaggc |
| | | SSIINFEKLDYKDDD | tcaggtggcggaggt |
| | | DK | tctgagcagaagttg |
| | | | atttcagaggaagat |
| | | | ctgagtattataaac |
| | | | ttcgagaagctgggt |
| | | | gggggaggaagttca |
| | | | atcattaattttgaa |
| | | | aaacttggaggaggt |
| | | | ggatcttccataatt |
| | | | aattttgaa |
| OVA³²³⁻³³⁹ | ISQAVHAAHAEINEA | GGGGSGGGGSEQKLI | agttgatttcagagg |
| | GR | SEEDLISQAVHAAHA | aagatctgatttcac |
| | | EINEAGRDYKDDDDK | aggctgtgcaggagg |
| | | | tggaggctcaggtgg |
| | | | cggaggttctgagca |
| | | | gatgctgcacatgct |
| | | | gaaattaatgaggct |
| | | | ggacgtgattataag |
| | | | gatgacgacgataag |
| | | | tga |
| Adpgk | ASMTNMELM | GGGGSGGGGSEQKLI | ggaggtggaggctca |
| | | SEEDLASMTNMELMG | ggtggcggaggttct |
| | | GGGSASMTNMELMGG | gagcagaagttgatt |
| | | GGSASMTNMELMDYK | tcagaggaagatctg |
| | | DDDDK | gctagcatgacgaac |
| | | | atggagctaatggga |
| | | | gggggcggaagtgct |
| | | | tctatgactaatatg |
| | | | gaactgatgggaggt |
| | | | ggaggttctgcttca |
| | | | atgaccaacatggaa |
| | | | cttatggattataag |
| | | | gatgacgacgataag |
| | | | tga |
| Rpl18 | KILTFDRL | GGGGSGGGGSEQKLI | ggaggtggaggctca |
| | | SEEDLKILTFDRLGG | ggtggcggaggttct |
| | | GGSKILTFDRLGGGG | gagcagaagttgatt |
| | | SKILTFDRLDYKDDD | tcagaggaagatctg |
| | | DK | aagattctgacgttt |
| | | | gatcgtctggggggg |
| | | | gcggatctaagatat |
| | | | tgacgttcgatcgac |
| | | | tgggaggaggtggct |
| | | | ctaagatcctaacat |
| | | | tcgaccgtctagatt |
| | | | ataaggatgacgacg |
| | | | ataagtga |
| P15E | KSPWFTTL | GGGGSGGGGSEQKLI | ggaggtggaggctca |
| | | SEEDLKSPWFTTLGG | ggtggcggaggttct |
| | | GGSKSPWFTTLGGGG | gagcagaagttgatt |
| | | SKSPWFTTLDYKDDD | tcagaggaagatctg |
| | | DK | aagtcaccttggttt |
| | | | actactctcggtggg |
| | | | ggaggaagtaaatcg |
| | | | ccatggtttactaca |
| | | | ttgggaggaggtggg |
| | | | tctaagtcaccatgg |
| | | | ttcacgacattggat |
| | | | tataaggatgacgac |
| | | | gataagtga |
| S21P2(1) | PSKPSKRSFIEDLLF | GGGGSGGGGSEQKLI | ggaggtggaggctca |
| | NKV | SEEDLPSKPSKRSFI | ggtggcggaggttct |
| | | EDLLFNKVDYKDDDD | gagcagaagttgatt |
| | | K | tcagaggaagatctg |
| | | | cctagtaagccttct |
| | | | aagcgcagcttcata |
| | | | gaagatttgttgttc |
| | | | aataaggtagattat |
| | | | aaggatgacgacgat |
| | | | aagtga |
| S21P2(2) | PSKPSKRSFIEDLLF | GGGGSGGGGSEQKLI | ggaggtggaggctca |
| | NKV | SEEDLPSKPSKRSFI | ggtggcggaggttct |
| | | EDLLFNKVGGGGSPS | gagcagaagttgatt |
| | | KPSKRSFIEDLLFNK | tcagaggaagatctg |
| | | VDYKDDDDK | cctagtaagccttct |
| | | | aagcgcagcttcata |
| | | | gaagatttgttgttc |
| | | | aataaggtaggtggg |
| | | | ggaggaagtccatcg |
| | | | aagccaagtaagcgt |
| | | | agtttcattgaggac |
| | | | ctcctcttcaacaag |
| | | | gttgattataaggat |
| | | | gacgacgataagtga |

### Example 2. Confirmation of Ability of Recombinant Reovirus with Introduced SARS-CoV-2 Epitope to Induce Production of SARS-CoV-2 Neutralizing Antibody

As an example of a reovirus-based vaccine platform, Reo-RBD (ReoV-RBD) was manufactured by fusing the codon-optimized SARS-CoV-2 receptor binding domain (RBD) to the σ1 protein of RP116 reovirus. That is, Reo-RBD has a gene with the SARS-CoV-2 receptor binding domain (RBD) base sequence introduced after the 251st codon of the S1 gene of RP116, whereby the SARS-CoV-2 receptor binding domain is created in a fused form with the σ1 protein. The present inventors expressed the recombinant reovirus in BHK21 cells and infected L929 cells with the obtained virus. The presence of the recombinant reovirus was detected using SARS-CoV-2 neutralizing antibody.

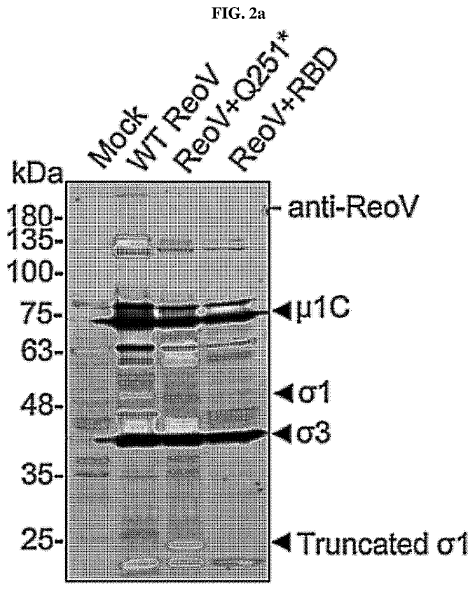

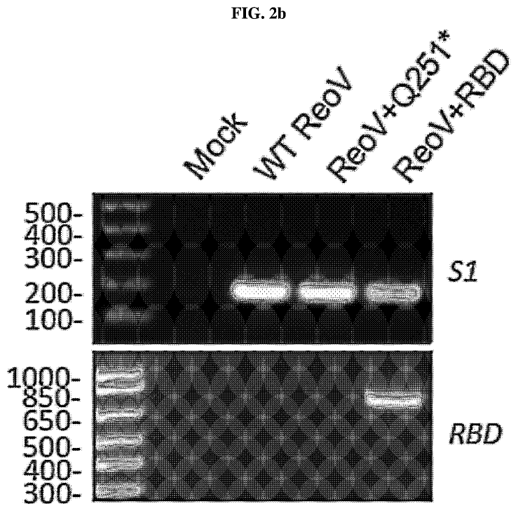

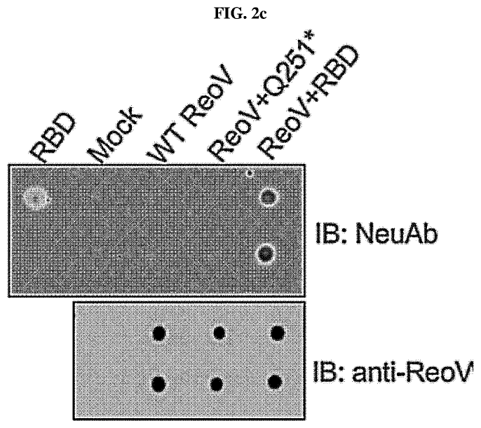

Specifically, the wild-type reovirus (WT ReoV), the mutant reovirus with truncated σ1 (ReoV+Q251*), and the recombinant reovirus (ReoV+RBD) introduced with the RBD antigen group were respectively infected into L929cell. After 96 hours later, cell lysates were analyzed. Recombinant RBD used as a positive control in this example was provided by the John Bell laboratory (Azad et al., 2020), and SARS-CoV-2 NeuAb (40592-MM57) was purchased from Sino Biological (Wayne, PA, USA). First, as a result of Western blot treatment with an anti-reovirus antibody, the reovirus protein was detected (FIG. 2a). In addition, after infecting L929cells with each recombinant reovirus, nucleic acid molecules were isolated from each cell, and RT-qPCR was performed using RBD gene-specific primers. As a result, the RBD gene was not detected in the cells infected with the wild-type reovirus or ReoV+Q251*, but the RBD gene was detected in cells infected with ReoV+RBD (FIG. 2b). In addition, as a result of performing dot blot analysis on the lysates of cells infected with each virus, reaction with anti-reovirus antibody (anti-ReoV) was confirmed in all groups (bottom of FIG. 2c), while the reaction with anti-SARS-CoV-1 neutralizing antibodies (NeuAb) was confirmed only in the lysate of cells infected with ReoV+RBD (top of FIG. 2c). The results demonstrate that recombinant Reo-RBD, in which the RBD gene is fused to the mutant S1 gene of RP116, has a structure recognizable by a neutralizing antibody against SARS-CoV-2.

### Example 3. Confirmation of Ability to Express Various Foreign Antigens of Reovirus-Based Vaccine Platform

Through the example, it was confirmed that the reovirus-based vaccine platform according to the present invention can react with the SARS-CoV-2 neutralizing antibody through the σ1 protein to which the SARS-CoV-2 RBD antigen group is fused. In this example, it was verified whether the vaccine platform of the present invention can express various foreign epitopes in addition to the antigen group.

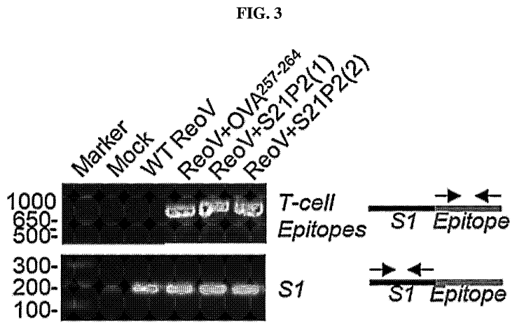

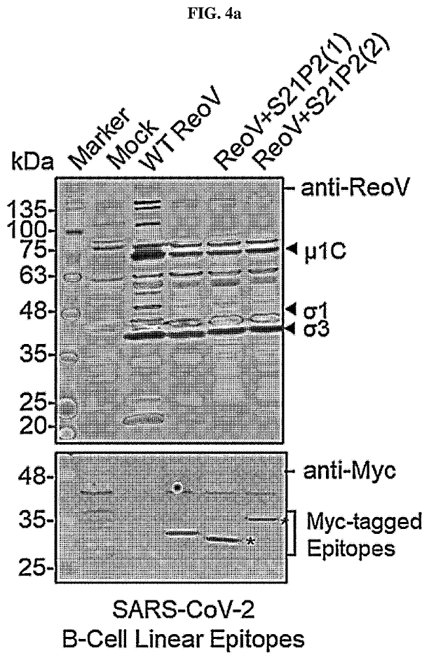

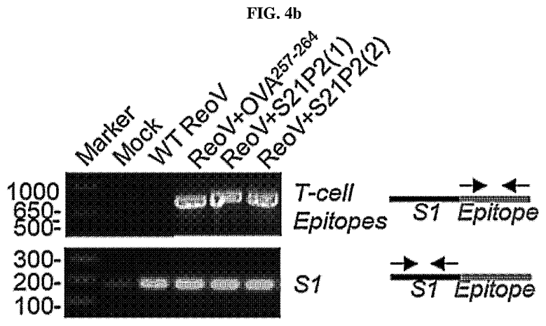

First, recombinant reoviruses fused with various CD4+ and CD8+ T cell epitopes (ovalbumin fragment, ADP-dependent glucokinase (Adpgk), ribosomal protein L18 (Rpl18)) were manufactured and infected into cells, and cell lysate was analyzed. As a result, reovirus-derived proteins were confirmed in each cell, and RNA of the S1 gene as well as the introduced T cell epitope RNA product were detected (FIG. 3). Also in cells infected with the recombinant reovirus containing the S1 gene fused to the B cell epitope (S21P2), it was confirmed that not only the reovirus-derived protein but also RNA of the introduced B cell epitope were expressed (FIGS. 4a and 4b).

The results show that the reovirus-based vaccine platform according to the present invention can express epitopes of various foreign antigens. Accordingly, the results suggest that epitopes of various diseases can be introduced into the present invention's vaccine platform and used for screening to develop new vaccines.

### Example 4. Confirmation of Cell Infection Ability and Foreign Epitope Expression Ability of Recombinant Reovirus Introduced with Ovalbumin Epitope

In this example, after producing the recombinant reovirus into which the ovalbumin (OVA) epitope was introduced, its ability to express foreign antigens was confirmed.

In the mutated S1 gene of the recombinant reovirus, the OVA257-264 epitope coupled with Myc tag and FLAG tag on each side thereof is fused. The Myc and FLAG tags can be detected by immunocytochemical analysis. Since the fluorescence of the tag protein overlaps with the fluorescence signal of the reovirus-derived protein, it indirectly indicates that the antigen of the foreign epitope is expressed in cells infected with the recombinant virus.

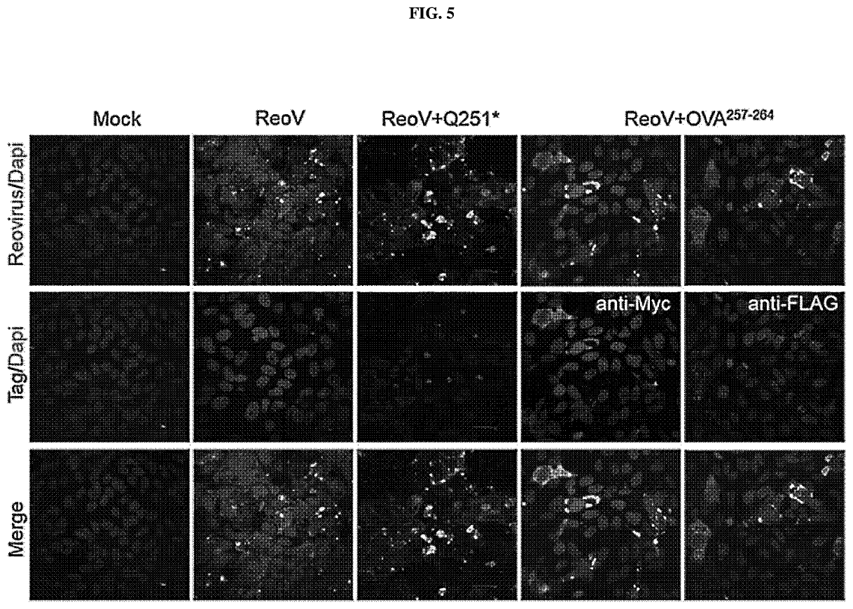

Specifically, Vero cells cultured on coverslips were infected with ReoV, ReoV+Q251*, or ReoV+OVA257-264 (recombinant reovirus into which OVA257-264 was introduced) for 48 hours. Next, the cells were fixed in 3.7% PFA and reacted with anti-ReoV (1:1000, rabbit serum), anti-Myc (1:200) and anti-FLAG (1:200) for 1 hour, and were treated with an anti-rabbit antibody labeled with Alexa Fluor 488 to detect anti-ReoV and with an antibody labeled with Alexa Fluor 594 to detect anti-Myc or anti-FLAG, and incubated for 45 minutes. After staining, the coverslips were mounted with Fluoromount G with DAPI for nuclear counterstaining. The samples were imaged using an Olympus confocal microscope with a 60× oil objective. As a result, fluorescent signals of tag proteins indicating the presence of OVA antigen were detected along with the fluorescent signal of the reovirus protein in cells infected with reovirus into which OVA was introduced, as shown in FIG. 5.

The result suggests that the reovirus-based vaccine platform according to the present invention is infectious to mammalian cells and can effectively express the introduced epitope.

### Example 5. Confirmation of Ability to Express Foreign Epitopes of Recombinant Reoviruses Introduced with T Cell Epitopes or SARS-CoV-2 Epitopes that Recognize Cancer Cells

Additionally, after constructing reoviruses into which a T cell epitope or SARS-CoV-2 epitope that recognizes cancer cells was introduced, it was verified whether the viruses induce the expression of the epitope in infected cells. Specifically, BHK21 cells were infected with the wild-type reovirus (WT); The recombinant reovirus (ReoV+p15E) expressing the T cell epitope p15E; or the reovirus (ReoV+S21P2(2)) expressing the SARS-CoV-2 epitope S21P2. Each epitope was tagged with Myc and FLAG. It was verified by Poh et al. (Nat. Comm., 2020) that the SARS-CoV-2 epitope induces the production of a neutralizing antibody against SARS-CoV-2. Western blot was performed with each cell lysate 48 hours after infection.

As a result, the reovirus proteins were detected in all samples, but Myc and FLAG tags were detected only in cells infected with the reovirus engineered with p15E or SARS-CoV-2 epitope. In addition, as a result of Western blotting using an anti-S2 antibody prepared using the entire protein of SARS-CoV-2 spike protein S2 (manufactured by Sino Biological) as an antigen, an immunoreactive protein band of the same size was identified only in the sample infected with ReoV+S21P2(2) reovirus.

These results suggest that the recombinant reoviruses possessing T cell epitope or SARS-CoV-2 epitope can induce specific immune responses against these epitopes.

### Example 6. Confirmation of Stability of Reovirus-Based Vaccine Platform

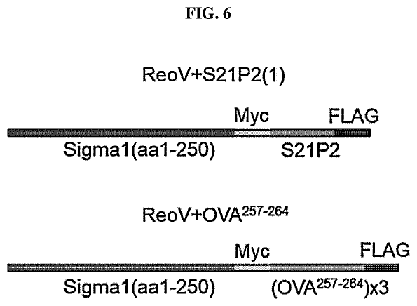

In this example, the stability of the reovirus-based vaccine platform according to the present invention was confirmed. For this, recombinant reoviruses expressing the S1 protein into which the S21P2 epitope or OVA257-264 was introduced were produced (FIG. 6). Each epitope was designed to contain a Myc or FLAG tag at each of the N-terminus and C-terminus thereof, and it was investigated whether the epitopes were stably expressed within cells through a reovirus.

Specifically, BHK21 cells were infected with the recombinant reovirus for 48 hours, and then Western blot was performed with the anti-reovirus antibody (anti-ReoV) and the anti-FLAG antibody (anti-FLAG), and as a result, both the reovirus protein band and the FLAG band were detected in the cells infected with the recombinant reovirus. The bands were detected at very high intensity in cells that had been passaged five times, proving that the expression of foreign epitopes by the recombinant reovirus of the present invention occurs stably even after several cell passages.

### Example 7. Confirmation of Ability of Recombinant Reovirus with Introduced SARS-CoV-2 Epitope to Induce Production of SARS-CoV-2 Neutralizing Antibody

Through the above example, the ability of the reovirus-based vaccine platform according to the present invention to express foreign antigens and the ability to induce the production of neutralizing antibodies against the antigens were confirmed. In this example, the ability of the vaccine platform to induce neutralizing antibodies was confirmed in vivo. As a representative example of a recombinant reovirus, a recombinant reovirus into which the SARS-CoV-2 epitope S21P2 was introduced was used.

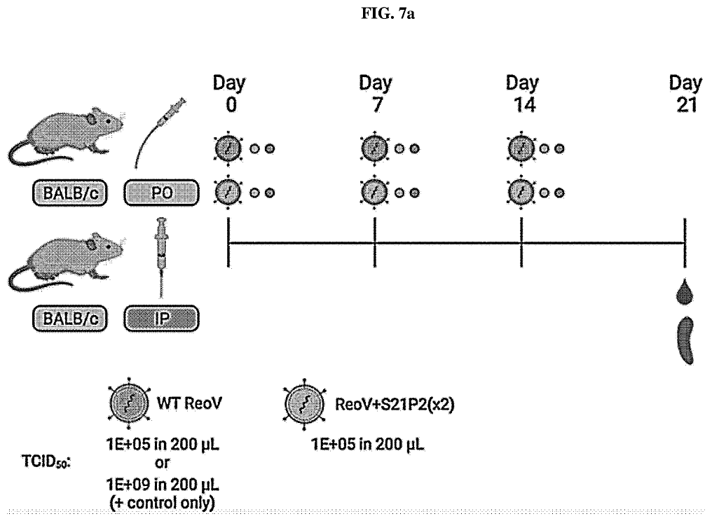

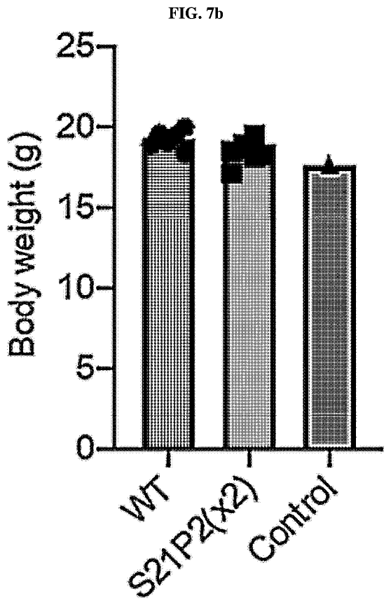

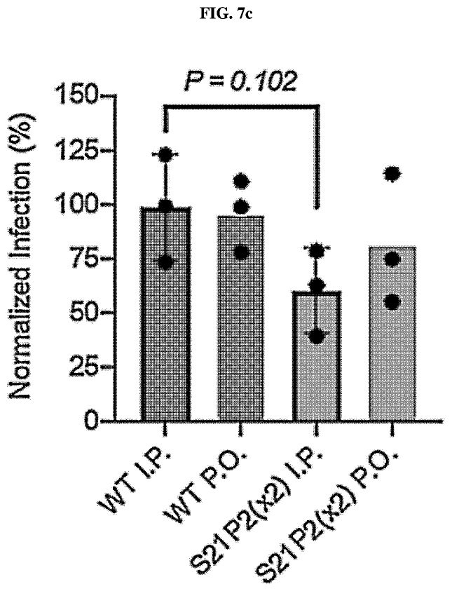

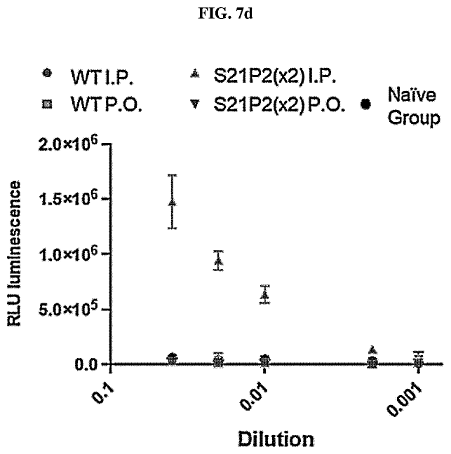

FIG. 7a is a schematic diagram showing the administration dose, route, and schedule of the recombinant reovirus-based vaccine. The reovirus-based vaccine was administered three times at one-week intervals by an oral or intraperitoneal route, and after one week, the mice were sacrificed and autopsied, and serum and spleen were used for analysis. As a result of comparing the body weight of each group on the 21st day of administration, which was the end of the experiment, no significant difference in body weight was observed (FIG. 7b). In addition, a neutralizing antibody was isolated from serum obtained from the mice and then treated with SARS-CoV-2-like viruses on HEK293T cells (n=2) to determine whether the neutralizing antibody prevents cell infection by SARS-CoV-2-like viruses. As a result, it was confirmed that the SARS-CoV-2-like virus was neutralized in the group treated with the neutralizing antibody isolated from the serum of mice inoculated with the S21P2 epitope-expressing reovirus vaccine, and the infection ability against ACE2-expressing HEK293T cells was reduced compared to the control group (FIG. 7c). In addition, as a result of ELISA analysis of immunoglobulins that specifically bind to the SARS-CoV-2 spike protein, a high level of fluorescence was observed in the serum of mice administered with the reovirus expressing the SARS-CoV-2 epitope. As a result, it was confirmed that an antibody against the SARS-CoV-2 spike protein exists (FIG. 7d). The results show that the recombinant reovirus into which the SARS-CoV-2 antigen is introduced can effectively induce the production of the SARS-Cov-2 neutralizing antibody in the mouse body, and accordingly, it can be used as a preventive vaccine against SARS-CoV-2 virus infection.

### Example 8. Confirmation of Immune Cell Activation Function of Reovirus-Based Vaccine

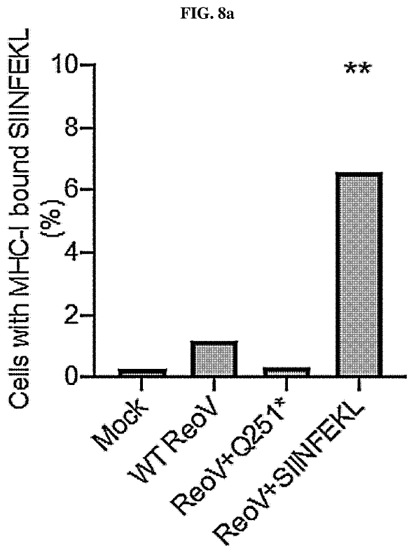

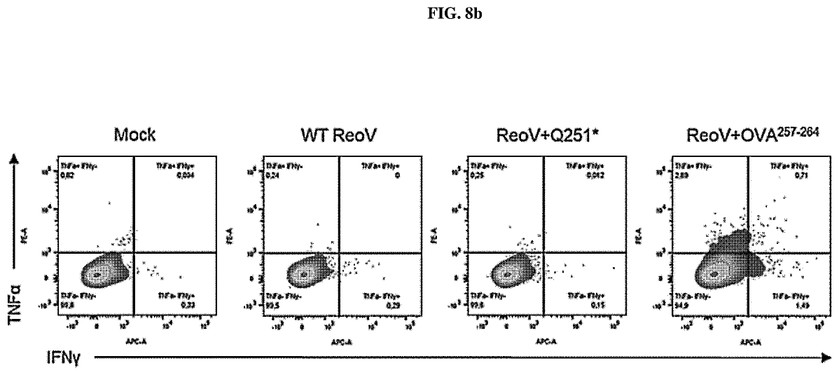

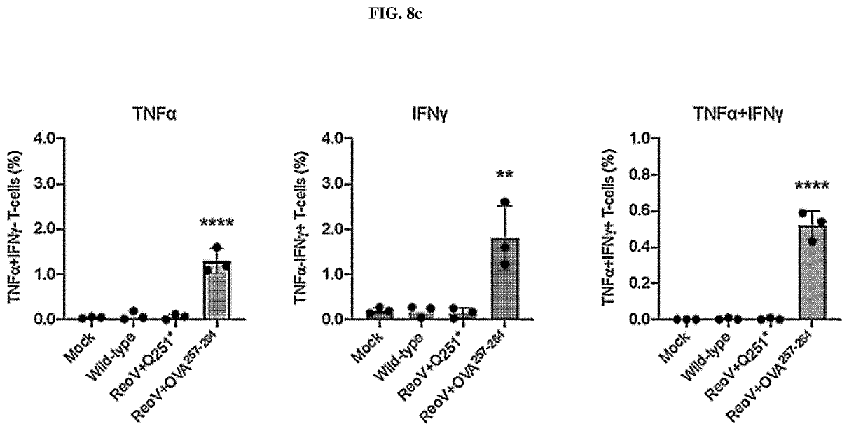

In this example, it was confirmed whether a reovirus-based vaccine introduced with a foreign epitope activates immune cells. First, as a representative example, a reovirus into which the ovalbumin epitope (OVA257-264) was introduced was manufactured and infected into cells, and it was confirmed whether the epitope was presented as major histocompatibility complex-I (MHC-I). L929-H2Kb cells infected with reovirus were collected and flow cytometry was performed with an antibody that can detect OVA257-264 bound to MHC-I. As a result, it was confirmed that the OVA257-264 epitope transmitted by reovirus was presented as MHC-I (FIG. 8a). Next, to check the immune cell activation function of the reovirus, L929-H2JKB cells were infected with wild-type reovirus (WT ReoV), a vaccine platform without an epitope (ReoV+Q257*), or reovirus introduced with OVA257-264 (ReoV+OVA257-264), and then co-cultured with OT-I mouse splenocytes. Cells whose culturing had been completed were collected and flow cytometry was performed using various T cell protein-specific antibodies and intracellular cytokine-specific antibodies. Results are shown in FIGS. 8b and 8c. As can be seen in the drawings, the proportion of CD8+ T cells expressing IFNγ and TNFα increased in cells infected with reoviruses into which the ovalbumin epitope was introduced, compared to cells infected with other reoviruses, indicating that ovalbumin epitope-specific T cells were activated. The results show that the reovirus-based vaccine of the present invention can infect cells and induce the expression of epitopes and antigen presentation by antigen-presenting cells, and accordingly, it can effectively induce the activation of the immune function of immune cells for the corresponding epitope.

### Example 9. Cancer Prevention and Treatment Mechanism of Reovirus-Based Vaccine Platform of Present Invention

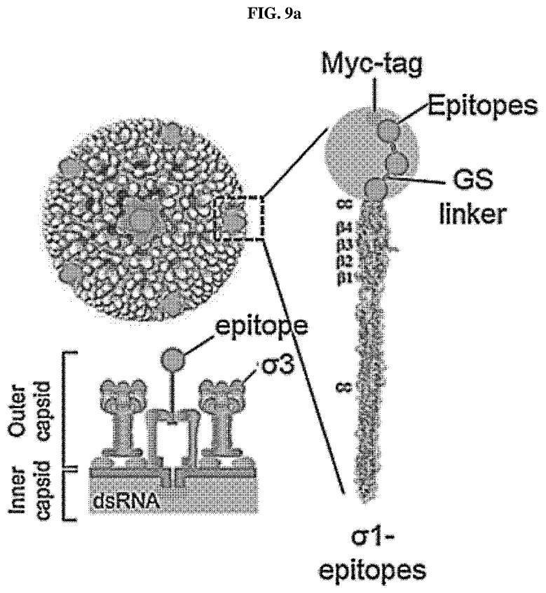

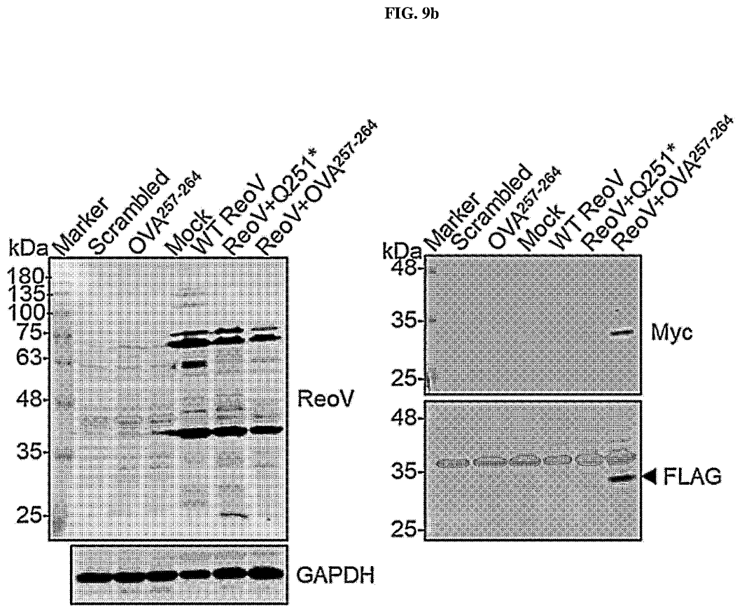

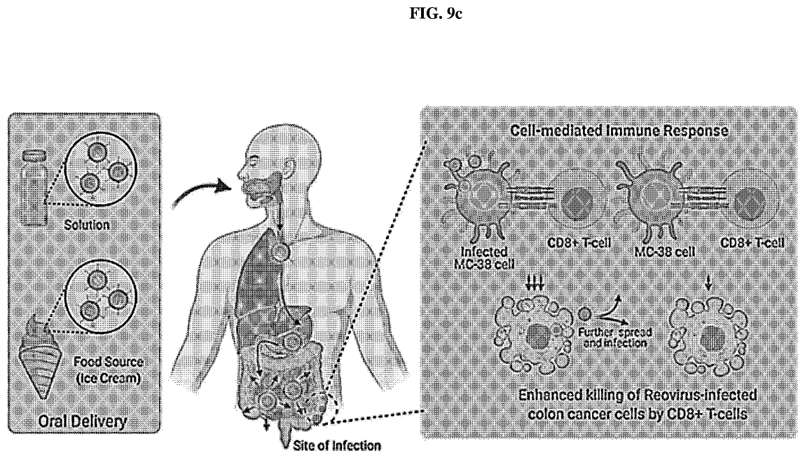

The reovirus-based vaccine platform according to the present invention can also be used for the prevention and treatment of cancer. For example, a recombinant reovirus that repeatedly expresses a tumor epitope can be produced by fusing the tumor epitope to the σ1 protein of the reovirus RP116 of the present invention (FIG. 9a). The stable cell infection ability and foreign epitope expression ability of the recombinant reovirus of the present invention were confirmed through several experiments. For example, after infecting L929 cells with various recombinant reoviruses for 72 hours, proteins from cell lysates thereof were analyzed. As a result, reovirus-derived proteins were detected in all experimental groups, and in cells infected with reovirus with an ovalbumin epitope, proteins tagged with the epitope were detected (FIG. 9b). Accordingly, It is also possible to manufacture recombinant reovirus that stably expresses a tumor antigen, and the reovirus can be used as a vaccine to prevent cancer. For example, colorectal cancer is a cancer that is difficult to diagnose in the early stages of development, but is located in an anatomical area that is easily accessible to reovirus. Accordingly, when a reovirus vaccine containing a colon cancer tumor antigen is administered to an individual, the vaccine exposes the tumor epitope to the body even before cancer develops, thereby inducing the body's preventive immune function against the tumor antigen. Additionally, even after the tumor has grown, it can induce an immune response that suppresses tumor growth. Accordingly, the reovirus-based vaccine platform of the present invention can be used as a material for the prevention and treatment of various diseases, including cancer, as well as infectious diseases caused by various viruses, bacteria, and fungi (FIG. 9c).

### Example 10. Confirmation of Cancer Prevention Effect of Reovirus-Based Vaccine

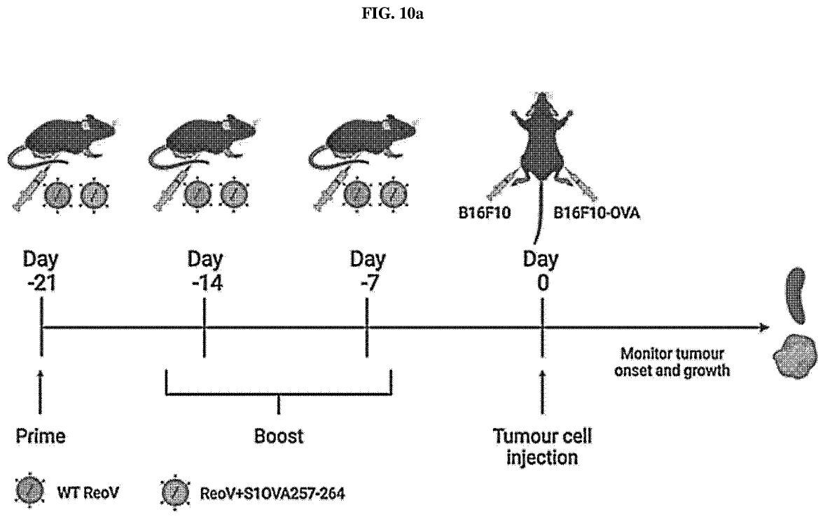

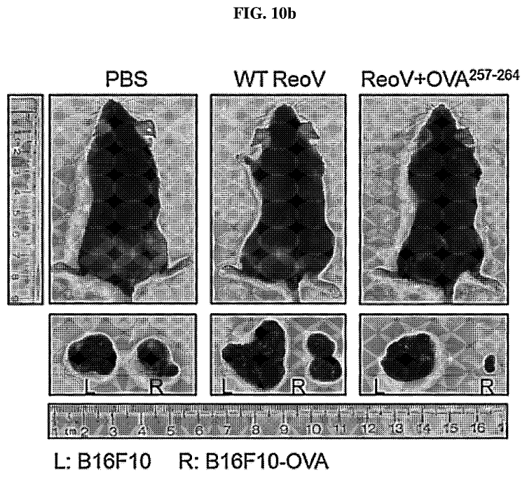

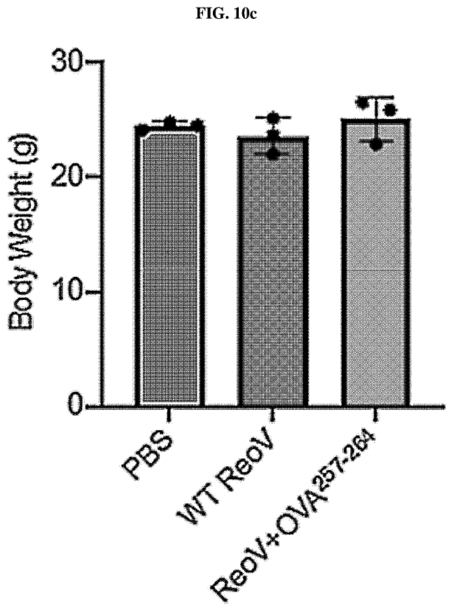

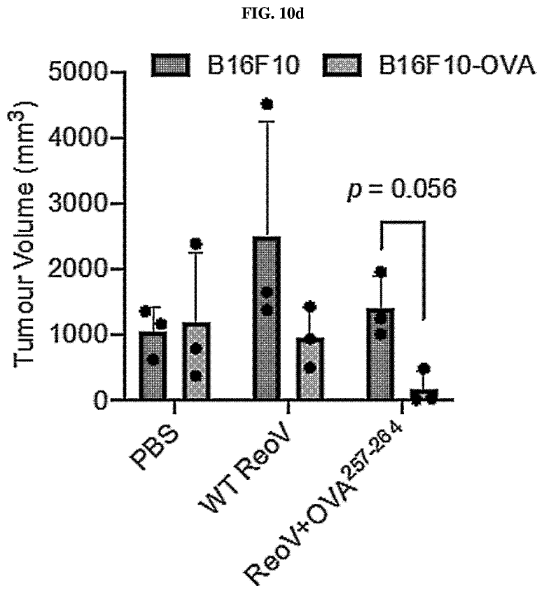

In this example, the cancer prevention effect of the reovirus-based vaccine was confirmed in vivo. For this, wild-type reovirus or reovirus introduced with ovalbumin antigen (S1OVA257-264) was administered to C57BL/6 mice through the intraperitoneal route a total of three times at weekly intervals, and after completing virus administration, mice were subcutaneously injected with 3×10⁶ B16F10 melanoma cells, and melanoma cells expressing ovalbumin-specific antigen (B16F10-OVA) on the left and right flanks, respectively (FIG. 10a). Next, tumor development and growth in mice were monitored and tumor size was measured using electronic calipers. When the tumor volume reached >1500 mm³ or vital signs reached euthanasia criteria, the mice were sacrificed and tumors were removed. As a result of comparing the tumor sizes of the euthanized mice, the B16F10-OVA tumor size was significantly reduced in the mice inoculated with the reovirus vaccine (ReoV+OVA257-264), into which OVA antigen was introduced, compared to the other groups. Accordingly, it was confirmed that the reovirus vaccine of the present invention effectively prevented tumor growth (FIG. 10b). For accurate verification, the sizes of tumors developed from normal B16F10 cells in each mouse were measured and compared with the sizes of tumors developed from B16F10 cells expressing an ovalbumin-specific antigen. As a result, in the mice inoculated with reovirus into which ovalbumin antigen was introduced, the size of tumors expressing ovalbumin-specific antigen was significantly reduced, compared to general tumors, and the size of ovalbumin antigen-expressing tumors was significantly reduced, compared to mice inoculated with a control group and wild-type reovirus (FIG. 10d). Accordingly, it can be seen that the reovirus vaccine of the present invention effectively suppressed the growth of ovalbumin-expressing tumors by inducing enhanced immune function against ovalbumin antigen. In addition, as no significant difference in body weight was confirmed between the control group and the reovirus vaccine administration group, it was confirmed that the reovirus-based vaccine of the present invention was not toxic (FIG. 10c).

### Example 11. Confirmation of Cancer Treatment Effectiveness of Reovirus-Based Vaccine

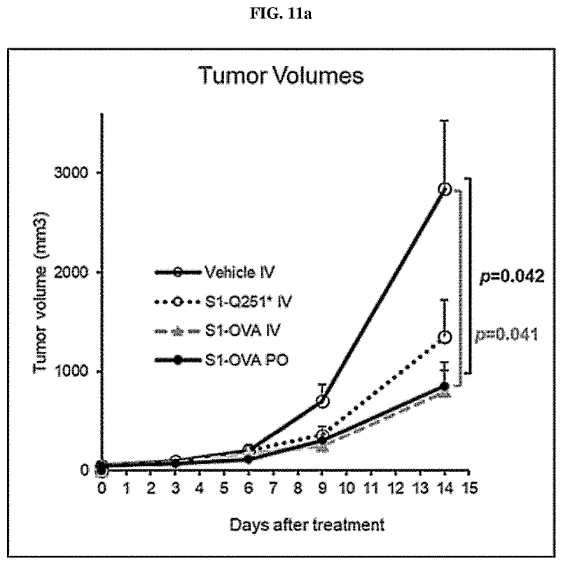

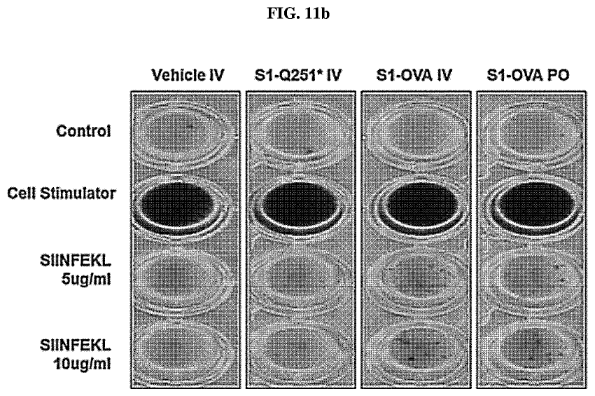

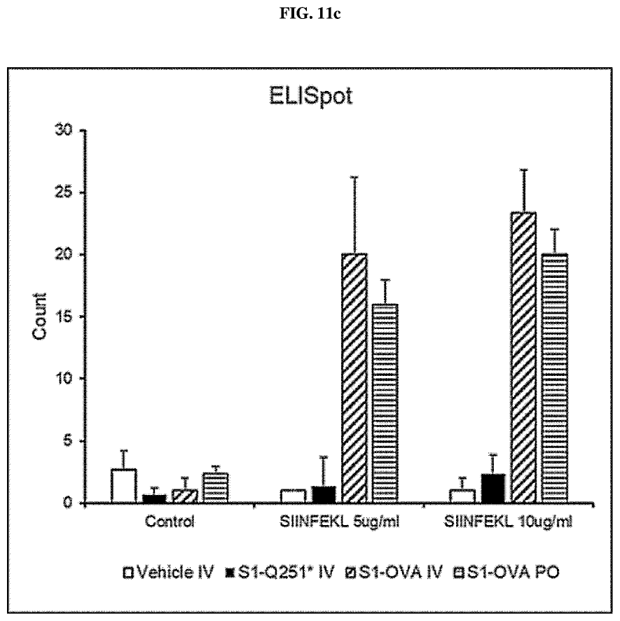

The cancer prevention effect of the reovirus-based vaccine of the present invention was confirmed through the above example. In this example, the cancer treatment effect of the vaccine was verified in vivo. First, 3×10⁵ melanoma B16F10-OVA cells expressing an ovalbumin epitope (OVA), a tumor cell-specific antigen, were subcutaneously injected into the flanks of C57BL/6 mice. Next, the onset and growth of the tumor were monitored and the size of the tumor was measured using electronic calipers. The recombinant reovirus vaccine expressing OVA-specific antigen was administered intravenously or orally three times on days 4, 11, and 18 after transplantation of the tumor cells. In addition, a vehicle, or reovirus (S1-Q251*) not expressing the OVA epitope was administered in the same amount as a control. When the tumor volume reached >2500 mm³ or vital signs reached euthanasia criteria, mice were sacrificed and tumors were removed. First, as a result of comparing tumor growth, the tumor growth of mice administered the recombinant reovirus into which the OVA epitope was introduced was noticeably delayed compared to other groups, confirming the tumor growth inhibition effect of the recombinant reovirus into which the epitope was introduced (FIG. 11a). In addition, CD8⁺ T cells isolated from mouse spleen cells were cultured and treated with ovalbumin peptide SIINFEKL to compare the activation levels of T cells. As a result, it was confirmed that T cells isolated from mice administered with the recombinant reovirus introduced with the OVA epitope were activated at a higher level by the antigen SIINFEKL than those in other groups (FIGS. 11b and 11c). It was found that the reovirus-based vaccine of the present invention effectively induced the immune function of anticancer immune cells against the tumor antigen. The results demonstrate that the reovirus-based vaccine of the present invention can effectively expose tumor epitopes to the body, activate immune function against tumor antigens, and significantly inhibit tumor growth.

The aforementioned description of the present invention is provided by way of example and those skilled in the art will understand that the present invention can be easily changed or modified into other specified forms without change or modification of the technical spirit or essential characteristics of the present invention. Therefore, it should be understood that the aforementioned examples are only provided by way of example and not provided to limit the present invention.

### INDUSTRIAL APPLICABILITY

The present invention relates to a novel reovirus-based vaccine platform, and confirmed that a part of the S1 gene of reovirus can be replaced with various exogenous epitope-encoding genes, and a recombinant reovirus manufactured according to the present invention not only can infect target cells and induce the expression of the epitope, but also can effectively prevent and treat diseases related to the epitope by activating the immune function of immune cells against the epitope. When using the reovirus-based vaccine platform of the present invention, vaccines containing various epitopes can be manufactured through relatively simple genetic manipulation technology, and can be administered in various ways including oral administration, so it can be utilized for the prevention and treatment of various infectious diseases including SARS-CoV-2 virus infection, and cancer.

1.-26. (canceled)

27. A recombinant vector, comprising a mutant S1 gene of reovirus and an exogenous epitope-encoding gene, wherein the mutant S1 gene has a stop codon at a 251st codon from a start codon thereof.

28. The recombinant vector according to claim 27, wherein the mutant S1 gene encodes a polypeptide comprising an amino acid sequence of SEQ ID NO: 1 or 2.

29. The recombinant vector according to claim 27, wherein the mutant S1 gene comprises one polynucleotide sequence selected from the group consisting of:
(a) a polynucleotide sequence of SEQ ID NO: 4 or 5; and
(b) a polynucleotide sequence of SEQ ID NO: 6 in which a 763rd nucleotide from a 5′ terminal is substituted with T.

30. The recombinant vector according to claim 27, wherein the exogenous epitope-encoding gene is located downstream of the mutant S1 gene.

31. The recombinant vector according to claim 27, wherein the mutant S1 gene and the exogenous epitope-encoding gene are expressed together to produce a fusion protein.

32. The recombinant vector according to claim 27, wherein the exogenous epitope is one or more selected from the group consisting of a viral antigen, a bacterial antigen, a fungal antigen, an allergen, and a tumor antigen.

33. The recombinant vector according to claim 32, wherein the virus is one or more selected from the group consisting of SARS-CoV-2 virus, norovirus, influenza virus, Ebola virus, human papillomavirus, hepatitis B virus, hepatitis C virus, hepatitis D virus, Marburg virus, human parainfluenza virus 1, measles virus, mumps virus, rabies virus, human immunodeficiency virus, dengue virus, poliovirus, cytomegalovirus, dengue virus, yellow fever virus, adenovirus, Japanese encephalitis virus, smallpox virus and Zika virus.

34. The recombinant vector according to claim 32, wherein the tumor antigen is one or more selected from the group consisting of ovalbumin, CD19, NY-ESO-1, EGFR, TAG72, IL13Rα2 (interleukin 13 receptor alpha-2 subunit), CD52, CD33, CD20, TSLPR, CD22, CD30, GD3, CD171, NCAM (neural cell adhesion molecule), FBP (folate binding protein), Le(Y) (Lewis-Y antigen), PSCA (prostate stem cell antigen), PSMA (prostate-specific membrane antigen), CEA (carcinoembryonic antigen), HER2 (human epidermal growth factor receptor 2), mesothelin, CD44v6 (hyaluronate receptor variant 6), B7-H3, glypican-3, ROR1 (receptor tyrosine kinase like orphan receptor 1), survivin, FOLR1 (folate receptor), WT1 (Wilm's tumor antigen), VEGFR2 (vascular endothelial growth factor 2), tumor virus antigen, TP53, and KRAS.

35. The recombinant vector according to claim 27, wherein the recombinant vector further comprises one or more genes selected from the group consisting of L1, L2, L3, M1, M2, M3, S2, S3, and S4.

36. A vaccine composition, comprising the recombinant vector according to claim 27, a mutant Sigma 1 protein expressed from the recombinant vector according to claim 27, a cell into which the recombinant vector has been introduced, or recombinant reovirus produced from the cell as an active ingredient.

37. The vaccine composition according to claim 36, wherein the protein is further fused with an exogenous epitope.

38. The vaccine composition according to claim 36, wherein the cell is further introduced with a vector comprising one or more genes selected from the group consisting of L1, L2, L3, M1, M2, M3, S2, S3, and S4 of reovirus.

39. The vaccine composition according to claim 36, wherein the recombinant reovirus comprises a mutant S1 gene and an exogenous epitope-encoding gene, and wherein the mutant S1 gene has a stop codon at a 251st codon from a start codon thereof.

40. The vaccine composition according to claim 39, wherein the reovirus expresses the exogenous epitope.

41. The vaccine composition according to claim 39, wherein the recombinant reovirus is produced from a cell into which the recombinant vector according to claim 27 has been introduced.

42. A method for prevention or treatment of viral diseases or cancer in a subject in need thereof, comprising:
administering to the subject a therapeutically effective amount of composition comprising a recombinant vector as an active ingredient, the recombinant vector comprising a mutant S1 gene of reovirus; and a viral epitope-encoding gene, or an epitope-encoding gene of a tumor antigen, and
wherein the mutant S1 gene has a stop codon at a 251st codon from a start codon thereof.

43. The method according to claim 42, wherein the virus is one or more selected from the group consisting of SARS-CoV-2 virus, norovirus, influenza virus, Ebola virus, human papillomavirus, hepatitis B virus, hepatitis C virus, hepatitis D virus, Marburg virus, human parainfluenza virus 1, measles virus, mumps virus, rabies virus, human immunodeficiency virus, dengue virus, poliovirus, cytomegalovirus, dengue virus, yellow fever virus, adenovirus, Japanese encephalitis virus, smallpox virus and Zika virus.

44. The method according to claim 42, wherein the tumor antigen is one or more selected from the group consisting of ovalbumin, CD19, NY-ESO-1, EGFR, TAG72, IL13Rα2 (interleukin 13 receptor alpha-2 subunit), CD52, CD33, CD20, TSLPR, CD22, CD30, GD3, CD171, NCAM (neural cell adhesion molecule), FBP (folate binding protein), Le(Y) (Lewis-Y antigen), PSCA (prostate stem cell antigen), PSMA (prostate-specific membrane antigen), CEA (carcinoembryonic antigen), HER2 (human epidermal growth factor receptor 2), mesothelin, CD44v6 (hyaluronate receptor variant 6), B7-H3, glypican-3, ROR1 (receptor tyrosine kinase like orphan receptor 1), survivin, FOLR1 (folate receptor), WT1 (Wilm's tumor antigen), VEGFR2 (vascular endothelial growth factor 2), tumor virus antigen, TP53, KRAS.

45. The method according to claim 42, wherein the cancer is one or more selected from the group consisting of squamous cell carcinoma, lung cancer, adenocarcinoma of lung, peritoneal cancer, skin cancer, melanoma, skin melanoma, intraocular melanoma, rectal cancer, anal cancer, esophageal cancer, small intestine cancer, endocrine cancer, parathyroid cancer, adrenal cancer, soft tissue sarcoma, urethral cancer, blood cancer, liver cancer, gastrointestinal cancer, pancreatic cancer, glioblastoma, cervical cancer, ovarian cancer, bladder cancer, liver cancer, breast cancer, colon cancer, colorectal cancer, endometrial or uterine cancer, salivary gland cancer, kidney cancer, prostate cancer, vulva cancer, thyroid cancer, head and neck cancer and brain cancer.

46. The method according to claim 42, wherein the composition is pharmaceutical or food composition.
